(19) United States
(12) Patent Application Publication
(10) Pub. No.: US 2020/0277379 A1
(43) Pub. Date: Sep. 3, 2020

(54) PHARMACEUTICAL COMPOSITION COMBINING IMMUNOLOGIC AND CHEMOTHERAPEUTIC METHOD FOR THE TREATMENT OF CANCER

(71) Applicant: Rampart Health, L.L.C., Orlando, FL (US)

(72) Inventors: David Granger BOSTWICK, Orlando, FL (US); Brian Rafferty BOSTWICK, Orlando, FL (US)

(21) Appl. No.: 16/804,733

(22) Filed: Feb. 28, 2020

Related U.S. Application Data
(60) Provisional application No. 62/812,703, filed on Mar. 1, 2019.

(51) Int. Cl. C07K 16/28; A61K 31/675; A61B 18/02; A61P 35/04

(52) U.S. Cl. CPC C07K 16/2818; C07K 16/2827; A61K 9/0019; A61B 18/02; A61P 35/04; A61K 31/675

(57) ABSTRACT

This invention relates to a pharmaceutical composition comprising at least two immune checkpoint inhibitors, at least one cytotoxic or cytostatic chemotherapeutic drug. This invention also relates to a method of treating a tumor or a cancer in a patient comprising administering to a patient in need thereof the pharmaceutical composition in an amount effective to treat the tumor or cancer, and optionally a step of ablating at least a portion of the tumor or cancer.

This application claims priority to U.S. Provisional Application No. 62/812,703, filed on Mar. 1, 2019, which is herein incorporated by reference in its entirety.

### FIELD OF THE INVENTION

This invention relates to pharmaceutical compositions combining immunologic and chemotherapeutic methods for the treatment of cancer.

### BACKGROUND

Cancer is the second most common cause of death in the US, claiming 580,000 Americans per year, more than 1,500 people each day. The National Institutes of Health (NIH) estimated the overall annual costs of cancer care at more than $227 billion (in 2007); including $89 billion for direct medical costs. Much of the overall healthcare costs of treating cancer are derived from management of the deleterious side effects of radiation and conventional chemotherapy. Immunologic cancer treatment is poised to completely change the landscape of oncologic therapeutics. Checkpoint inhibitors, such as CTLA-4 and PD-1, are already making a major impact in the treatment of metastatic melanoma and non-small cell lung cancer. These drugs are now being used in combination in an attempt to improve their efficacy. The delivery of these drugs is most commonly performed intravenously which can have serious and sometimes fatal systemic toxicities as a result of nonspecific distribution of these cytocidal agents in the body, which kill both cancer cells and normal cells and can negatively impact the treatment regimen and patient outcome.

Ablation is a surgical technique used to destroy cells, organs, or abnormal growths (such as cancers). Cryoablation has been known to illicit an immune response in patients through the presentation of a unique array of tumor associated antigens to a patient's antigen presenting cells and dendritic cells. This “cryoimmunologic effect”, however, has been known to be variable and in some instances even detrimental.

WO 2017/123981 relates to a pharmaceutical composition comprising at least two immune checkpoint inhibitors and at least one cytokine, and its combination with an ablation step. Cytokine is a naturally-occurring protein that is secreted by cells of the immune system or non-immune cells (e.g. epithelial cells) in response to a number of stimuli and assist in regulating the development of immune effector cells. Cytokine is an immunomodulation agent that acts through a mechanism that ultimately alters gene expression in the target cells. The combination of the two immune checkpoint inhibitors and a cytokine is within the regime of immunotherapy by using exclusive immunologic agents.

There thus remains a need in the art to develop an improved method to not only reduce the toxicities associated with traditional systemic cancer treatments but also provide an optimal cancer immune response for an improved treatment of cancers. This disclosure answers that need.

### SUMMARY OF THE INVENTION

One aspect of the invention relates to a pharmaceutical composition, comprising at least two immune checkpoint inhibitors, at least one cytotoxic or cytostatic chemotherapeutic drug. Optionally, the pharmaceutical composition can comprise a pharmaceutically acceptable carrier.

Another aspect of the invention relates to a method of treating a tumor or a cancer in a patient comprising: administering to a patient in need thereof a composition comprising: at least two immune checkpoint inhibitors and at least one cytotoxic or cytostatic chemotherapeutic drug, in an amount effective to treat the tumor or cancer. The method may further comprise a step of ablating at least a portion of the tumor or cancer.

Additional aspects, advantages and features of the invention are set forth in this specification, and in part will become apparent to those skilled in the art on examination of the following, or may be learned by practice of the invention. The inventions disclosed in this application are not limited to any particular set of or combination of aspects, advantages and features. It is contemplated that various combinations of the stated aspects, advantages and features make up the inventions disclosed in this application.

### BRIEF DESCRIPTION OF THE DRAWINGS

FIGS. 1A-1B are images of FDG-PET (¹⁸F-fluorodeoxyglucose positron emission tomography) scans of Patient A's whole body bone before (FIG. 1A) and about 3 months after (FIG. 1B) treatment with a combination of a CTLA-4 inhibitor, a PD-1 inhibitor, and a low-dose chemotherapeutic agent with a temperature-limited cryoablation procedure. Comparison of the scans before (FIG. 1A) and about 3 months after (FIG. 1B) treatment reveals considerable improvement in bone metastases; the arrows point to the region (the pelvis region) that the improvements are most prominent.

FIG. 2 is a graph showing the results of Patient B's serum prostate-specific antigen (PSA) concentrations following two rounds of treatments with a combination of a CTLA-4 inhibitor, a PD-1 inhibitor, and a low-dose chemotherapeutic agent with a temperature-limited cryoablation procedure. The stars indicate the dates of treatment. The graph shows a PSA decline from 107.6 to 31.9 ng/mL (70% decline) following two treatments.

### DETAILED DESCRIPTION OF THE INVENTION

The present disclosure is based, at least in part, on the development of new compositions and methods to illicit a cancer immune response through a combination of tumor-directed immunologic cancer treatments and ablation techniques. Intra-tumoral administration of these treatments and procedures may have significant advantages over traditional systemic delivery of anti-cancer drugs. The compositions and methods disclosed herein can allow for smaller than traditional doses to be administered to the subject (e.g., in embodiments wherein the compositions are administered directly into the tumor), a stimulation of the immune system against the tumor antigens, and improved results by placing the drugs in direct proximity to the tumor antigens and the immune inflammatory process.

The inventors surprisingly discovered that, by using the combination of immune checkpoint inhibitors and cytotoxic or cytostatic chemotherapeutic drugs, the treatment method provided at least the following benefits, including: (1) inducing immune-stimulating necrosis by minimally-invasive ablation; (2) preserving cancer neo-antigens by employing minimal thermal ablation; (3) safeguarding adjacent protein structures by limiting the size of the ablation; and (4) intra-tumorally injecting of combination immunotherapy combining with the ablation allows for a low-dose (lower than traditional doses) immunotherapy.

In one aspect, the present disclosure provides a pharmaceutical composition comprising, consisting essentially of, or consisting of, a combination of at least two immune checkpoint inhibitors and at least one cytotoxic or cytostatic chemotherapeutic drug. Optionally, the pharmaceutical composition can comprise a pharmaceutically acceptable carrier.

Immune checkpoint inhibitors are a type of drug that blocks certain proteins made by some types of immune system cells, such as T cells, and some cancer cells. These proteins help keep immune responses in check and can keep T cells from killing cancer cells. When these proteins are blocked, the “brakes” on the immune system are released and T cells are able to kill cancer cells better. Checkpoint inhibitors therefore work to activate the immune system to attack tumors, inhibiting the immune response proteins responsible for down regulating the immune system. Such checkpoint inhibitors may include small molecule inhibitors or may include antibodies, or antigen binding fragments thereof, that bind to and block or inhibit immune checkpoint receptors or antibodies that bind to and block or inhibit immune checkpoint receptor ligands.

For instance, PD-1 and CTLA-4 attenuate T-cell activity through independent molecular mechanisms. See Das et al., “Early B cell changes predict autoimmunity following combination immune checkpoint blockade.” J Clin Invest. 128(2):715-720 (2018); Das et al., “Combination therapy with anti-CTLA-4 and anti-PD-1 leads to distinct immunologic changes in vivo.” J Immunol. 194(3):950-959 (2015), which are incorporated by reference in their entirety. The enhanced benefit of combination checkpoint inhibitor blockade is likely mediated by multiple mechanisms distinct from the component monotherapies rather than by additive engagement of the cellular and molecular mechanisms of each monotherapy. See Wei et al., “Fundamental Mechanisms of Immune Checkpoint Blockade Therapy. Cancer Discov.” 8(9):1069-1086 (2018), which is incorporated by reference in its entirety. It is possible that positive co-stimulation by blockade beyond physiologic levels facilitates acquisition of enhanced cytolytic capabilities or novel properties not displayed by canonical T-cell populations, resulting in enhanced efficacy. Little has been known of the relative contribution for each of the several known molecular mechanisms of PD-1 and CTLA-4 blockade to therapeutic efficacy. Combination checkpoint inhibitor blockade therapy can improve therapeutic efficacy compared with monotherapy in both preclinical and clinical studies. See Curran et al., “PD-1 and CTLA-4 combination blockade expands infiltrating T cells and reduces regulatory T and myeloid cells within B16 melanoma tumors.” Proc Natl Acad Sci USA. 107(9):4275-4280 (2010); Postow et al., “Immunologic correlates of the abscopal effect in a patient with melanoma.” New England Journal of Medicine. 366(10):925-931 (2012); Wolchok et al., “Ipilimumab monotherapy in patients with pretreated advanced melanoma: a randomised, double-blind, multicentre, phase 2, dose-ranging study.” Lancet Oncol. 11(2):155-164 (2010), which are incorporated by reference in their entirety. Patients with metastatic melanoma treated by combination therapy with PD-1 and CTLA-4 blockade may achieve responses in 36% and greater than 50% in some instances, with 57% 3-year overall survival. See Larkin et al., “Combined Nivolumab and Ipilimumab or Monotherapy in Untreated Melanoma.” N Engl J Med. 373(1):23-34 (2015), which is incorporated by reference in its entirety. Combination therapy may also produce overall survival benefit in metastatic renal cell carcinoma when compared with standard-of-care. See Motzer et al., “Nivolumab plus Ipilimumab versus Sunitinib in Advanced Renal-Cell Carcinoma.” N Engl J Med. 378(14):1277-1290 (2018), which is incorporated by reference in its entirety.

The checkpoint inhibitors comprise inhibitors such as inhibitors of CD137 (4-1BB); CD134; PD-1; KIR; LAG-3; PD-L1; PDL2; CTLA-4; B7 family ligands such as B7.1 (or CD80) or B7.2 (or CD86), B7-DC, B7-H1, B7-H2, B7-H3 (or CD276), B7-H4, B7-H5, B7-H6 and B7-H7; BTLA (or CD272); LIGHT; HVEM; GALS; TIM-3; TIGHT; VISTA; 2B4; CGEN-15049; CHK1; CHK2; A2aR; TGF-β; PI3Kγ; GITR; ICOS; IDO; TLR; IL-2R; IL-10; PVRIG (B7/CD28); CCRY; OX-40; CD160; CD20; CD52; CD47; CD73; CD27-CD70; and/or CD40.

Suitable CD137 (4-1BB) inhibitors include, but are not limited to, utomilumab, urelumab, or a combination thereof. Suitable CD134 or OX40 inhibitors include, but are not limited to, OX40-immunoglobulin (OX40-Ig), GSK3174998 (an anti-OX40 antibody), 9B12, MOXR 0916, PF-04518600 (PF-8600), MEDI6383, MEDI0562, INCAGN01949, or a combination thereof. Suitable KIR inhibitors include, but are not limited to, IPH4102, 1-7F9 (a human monoclonal antibody that binds KIR2DL1/2L3), lirilumab, or a combination thereof. Suitable LAG-3 inhibitors include, but are not limited to, relatlimab, IMP321 (Immuntep®), GSK2831781 (an agonist antibody to LAG3), BMS-986016, LAG525, or a combination thereof. Suitable CTLA-4 inhibitors include, but are not limited to, ipilimumab, tremelimumab, or a combination thereof. Suitable PD-1 inhibitors include, but are not limited to, pembrolizumab, nivolumab, pidilizumab, MK-3475, MED 14736 (a monoclonal antibody), CT-011, spartalizumab, or a combination thereof. Suitable PD-L1 or PD-L2 inhibitors include, but are not limited to durvalumab, atezolizumab, avelumab, AMP224, BMS-936559, MPLDL3280A (an anti-PD-L1 antibody), MSB0010718C (an anti-PD-L1 antibody), or a combination thereof. Suitable B7.1 (or CD80) or B7.2 (or CD86) inhibitors include, but are not limited to, rhudex, abatacept, or a combination thereof. Suitable B7-H3 inhibitors include, but are not limited to, enoblituzumab (MGA271), MGD009, 8H9 (a monoclonal antibody to B7-H3), or a combination thereof. Suitable CD20 inhibitors include, but are not limited to rituximab, ofatumumab, or a combination thereof. Suitable CD52 inhibitors include, but are not limited to alemtuzumab. Suitable CD47 inhibitors include, but are not limited to, Hu5F9-G4, TTI-621 (SIRPaFc), or a combination thereof. Suitable CD73 inhibitors include, but are not limited to, MEDI9447. Suitable CD27-CD70 inhibitors include, but are not limited to, ARGX-110, BMS-936561 (MDX-1203), varilumab, or a combination thereof. Suitable CD40 inhibitors include, but are not limited to, CP-870893, APX005M, ADC-1013, JNJ-64457107, SEA-CD40, R07009789, or a combination thereof.

Suitable BTLA (or CD272) inhibitors include, but are not limited to 40E4; 40E4 mIgG1; D265A, or a combination thereof. Suitable LIGHT (or CD272) inhibitors include, but are not limited to T5-39; 17-2589-42 (a CD258 (LIGHT) monoclonal antibody), TNFSF14, or a combination thereof. Suitable HVEM inhibitors include, but are not limited to anti-CD270. Suitable TIM-3 inhibitors include, but are not limited to MBG453, MEDI9447, or a combination thereof. Suitable TIGHT inhibitors include, but are not limited to, OMP-31M32. Suitable VISTA inhibitors include, but are not limited to, JNJ-61610588, CA-170, or a combination thereof. Suitable CGEN-15049 inhibitors include, but are not limited to, anti-CGEN-15049. Suitable A2aR inhibitors include, but are not limited to, CPI-444. Suitable TGF-β inhibitors include, but are not limited to, trabedersen (AP12009), M7824, galusertinib (LY2157299), or a combination thereof. Suitable PI3Kγ inhibitors include, but are not limited to, IPI-549. Suitable GITR inhibitors include, but are not limited to, TRX-518, BMS-986156, AMG 228, MEDI1873, MEDI6469, MK-4166, INCAGN01876, GWN323, or a combination thereof. Suitable ICOS inhibitors include, but are not limited to, JTX-2011, GSK3359609, MEDI-570, or a combination thereof. Suitable IDO inhibitors include, but are not limited to, BMS-986205, indoximod, epacadostat, or a combination thereof. Suitable TLR inhibitors include, but are not limited to, MEDI9197, PG545 (pixatimod, pINN), polyinosinic-polycytidylic acid polylysine, carboxymethylcellulose (poly-ICLC), or a combination thereof. Suitable IL-2R inhibitors include, but are not limited to, NKTR-214. Suitable IL-10 inhibitors include, but are not limited to, AM0010. Suitable PVRIG (B7/CD28) inhibitors include, but are not limited to, COM701.

Additional checkpoint inhibitors suitable for use herein also include those described in Marin-Acevedo et al., “Next generation of immune checkpoint therapy in cancer: new developments and challenges,” Journal of Hematology & Oncology 11:39 (2018), which is incorporated herein by reference in its entirety.

The pharmaceutical composition can comprise any combination of two or more check point inhibitors. They may be the same type of checkpoint inhibitors or they may be different checkpoint inhibitors. In some embodiments, the at least two checkpoint inhibitors comprise a CTLA-4 inhibitor and a PD-1 inhibitor. In some embodiments, the at least two checkpoint inhibitors comprise a CTLA-4 inhibitor and a PD-L1 inhibitor. In some embodiments, the CTLA-4 inhibitor is ipilimumab and the PD-1 inhibitor is pembrolizumab or nivolumab.

A skilled practitioner would appreciate that many other combinations of the checkpoint inhibitors are also suitable for the pharmaceutical composition. A non-limiting list of the combinations include a CD137 inhibitor and a CD134 inhibitor; a PD-1 inhibitor and a KIR inhibitor; a LAD-3 inhibitor and a PD-L1 inhibitor; a CTLA-4 inhibitor and a CD40 inhibitor; a CD 134 inhibitor and a PD-1 inhibitor; a KIR inhibitor and a LAG-3 inhibitor; a PD-L1 inhibitor and a CTLA-4 inhibitor; a CD40 inhibitor and a CD 137 inhibitor; a CTLA-4 inhibitor and a PD-L1 inhibitor; a PD-1 inhibitor and a CD40 inhibitor; or any other combinations of two or more of the checkpoint inhibitors known in the art.

The pharmaceutical compositions can also comprise at least one cytotoxic or cytostatic chemotherapeutic drug. The term “cytotoxic” or “cytostatic” refers to a cellular component or a drug that can cause the inhibition of cell growth and multiplication of cancer cells or cause cancer cells to die.

Suitable cytotoxic or cytostatic chemotherapeutic drugs include, but are not limited to, actinomycin, aldesleukin, alemtuzumab, alitretinoin, altretamine, amsacrine, anastrozole, arsenic trioxide, asparaginase, azacitidine, azathioprine, bacillus calmette-geurin vaccine (BCG), bevacizumab, bexarotene, bicalutamide, bleomycin, bortezomib, botulinum toxin (Botox), busulfan, capecitabine, carboplatin, carmustine, cetrorelix acetate, cetuximab, clorambucil, chloramphenicol, chlormethine hydrochloride, choriogonadotropin alfa, ciclosporin, cidofovir, cisplatin, cladribine, clofarabine, clorambucil, colchicine, crisantaspase, cyclophosphamide, cytarabine, dacarbazine, dactinomycin, danazol, dasatinib, daunorubicin HCl, decitabine, denileukin, dienostrol, diethylstilbestrol, dinoprostone, dithranol-containing products, docetaxel, doxorubicin, dutasteride, epirubicin, ergometrine/methylergometrine, estradiol, estramustine phosphate sodium, estrogen-progestin combinations, conjugated estrogens, esterified estrogens, estrone, estropipate, etoposide, exemestane, finasteride, floxuridine, fludarabine, fluorouracil, fluoxymesterone, flutamide, fulvestrant, ganciclovir, ganirelix acetate, gemcitabine, gemtuzumab ozogamicin, gondaotrophin, chorionic goserelin (zoladex), hydroxycarbamide, ibritumomab tiuxetan, idarubicin, ifosfamide, imatinib mesilate, interferon Alfa-2b, interferon-containing products, irinotecan HCl, leflunomide, letrozole, leuprorelin acetate, lomustine, lymphoglobuline, medroxyprogesterone, megestrol, melphalan, menotropins, mercaptopurine, mesena, methotrexate, methyltestosterone, mifepristone, mitomycin, mitotane, mitoxantrone HCl, mycophenolate, mofetil, nafarelin, natalizumab, nilutamide, oestrogen-containing products, oxaliplatin, oxytocin (including syntocinon and syntometrine), paclitaxel, paraldehyde, pegaspargase, pemetrexed disodium, pentamidine isethionate, pentostatin, perphosphamide, pipobroman, piritrexim isethionate, plicamycin, podoflilox, podophyllin, podophyllum resin, prednimustine, procarbazine, progesterone-containing products, progestins, raloxifene, raltitrexed, ribavirin, rituximab, sirolimus, streptozocin, tacrolimus, tamoxifen, temozolomide, teniposide, testolactone, testosterone, thalidomide, thioguanine, thiotepa, thymoglobulin, tioguanine, topotecan, toremifene citrate, tositumomab, trastuzumab, treosulfan, tretinoin, trifluridine, trimetrexate glucoronate, triptorelin, uramustine, vaccines (live), valganciclovir, valrubicin, vidarabine, vinblastine sulfate, vincristine, vindesine, vinorelbine tartrate, zidovudine, or a combination thereof. Exemplary cytotoxic or cytostatic chemotherapeutic drugs are asparaginase, bleomycin, busulphan, carboplatin, cetuximab, cisplatin, cyclophosphamide, BCG, chloramphenicol, colchicine, cyclosporin, dacarbazine, doxorubicin, etoposide, fludarabine, gemcitabine, ifosfamide, irinotecan, lomustin, melphalan, methotrexate, mitomycin, mitoxantrone, paclitaxel, procarbazine, rituximab, temozolomide, thitepa, vinblastine, vincristine, zidovudine, and a combination thereof. The combination of two or more check point inhibitors with a chemotherapeutic agent (cytostatic or cytotoxic) is different than the combination of two or more check point inhibitors with another immunotherapeutic agent, such as a cytokine. Fundamentally, the drug classes for and mechanism of action in the polypharmacy combinations of the former combination differ from those of the latter combination. In particular, chemotherapeutic agents are usually anti-metabolites and are synthetic drugs, not protein drugs, whereas cytokines are naturally-occurring proteins and are considered biologics. Although both classes of these agents have pleiotropic effects on the immune system, the repertoire of effects and the mechanisms of actions to induce these effects are markedly different for these two different classes of agents. Additionally, the mechanism of suppression of cytokines (suppressor of cytokine signaling proteins) differs from that of chemotherapeutic drugs.

Cytokines are low molecular weight regulatory proteins or glycoproteins that are usually secreted by cells of the immune system or non-immune cells (e.g. epithelial cells) in response to a number of stimuli and assist in regulating the development of immune effector cells. Cytokines bind to the specific receptors on the membrane of target cells, triggering signal transduction pathways that ultimately alter gene expression in the target cells. The actions of cytokines are involved in a wide range of biological processes. On the other hand, chemotherapeutic agents may promote cancer immunity by inducing immunogenic cell death directly or indirectly. Direct actions of chemotherapy include induction of necroptosis or autophagy. Indirect actions include altering cell signaling pathways, thwarting efforts used by cancer to avoid immune modulation (see Emens et al., “Chemotherapy: friend or foe to cancer vaccines?” Curr Opin Mol Ther. 3(1):77-84 (2001), which is incorporated herein by reference in its entirety); release and enhancement of presentation of cancer neoantigens and danger-associated molecular patterns (DAMP), such as, for example, when chemokine signaling by CXCL8 stimulates dendritic cell identification and consumption of injured cancer cells by exposing calreticulin on the cell surfaces (see Sukkurwala et al., “Immunogenic calreticulin exposure occurs through a phylogenetically conserved stress pathway involving the chemokine CXCL8.” Cell Death Differ. 21(1):59-68 (2014), which is incorporated herein by reference in its entirety); enhancement of effector T-cell activity by upregulating MHC class 1 expression, costimulatory molecules such as B7-1, or the cancer neoantigens themselves; or by downregulating coinhibitory molecules such as PD-L1/B7-H1 or B7-H4 (see Chen et al., “Chemoimmunotherapy: reengineering tumor immunity.” Cancer Immunol Immunother. 62(2):203-216 (2013), which is incorporated herein by reference in its entirety). Chemotherapy-induced T-cell mediated killing of cancer may involve fas-, perforin-, and Granzyme B-dependent mechanisms. See Chen et al., “Chemoimmunotherapy: reengineering tumor immunity.” Cancer Immunol Immunother. 62(2):203-216 (2013), which is incorproated herein by reference in its entirety.

Cytostatic and cytotoxic chemotherapeutic agents alone have shown dose-dependent effects on the immune system. See Emens, “Chemoimmunotherapy.” Cancer J. 16(4):295-303 (2010); Chen et al., “Chemoimmunotherapy: reengineering tumor immunity.” Cancer Immunol Immunother. 62(2):203-216 (2013), which are incorporated by reference in their entirety.

The chemotherapeutic agents have been used to regulate cancer immunity while avoiding the toxicity associated with higher doses required for direct cell killing. This modulation has been demonstrated with several chemotherapeutic agents, such as cyclophosphamide, paclitaxel, cisplatin, and temozolomide. For example, cyclophosphamide has shown pleiotropic immune-modulating properties, including, e.g., depleting Tregs. See Machiels et al., “Cyclophosphamide, doxorubicin, and paclitaxel enhance the antitumor immune response of granulocyte/macrophage-colony stimulating factor-secreting whole-cell vaccines in HER-2/neu tolerized mice.” Cancer Res. 61(9):3689-3697 (2001), which is incorporated by reference in its entirety. Taxanes such as paclitaxel may also deplete Tregs, facilitate dendritic cell maturation, and shift the CD4+ T-helper phenotype from type 2 to type 1, resulting in enhanced proinflammatory cytokine secretion and priming and lytic activity of CD8+ T cells. Doxorubicin may delay tumor outgrowth and enhance vaccine activity, although the mechanism of this immunomodulation is uncertain. Combination of cyclophosphamide and doxorubicin have also shown favorable effect, curing some mice of cancer with selective depletion of Tregs, allowing recruitment of high-avidity cancer-specific T cells. Combination of a HER2p, GM-CSF-secreting breast cancer vaccine, with immune-modulating doses of cyclophosphamide and doxorubicin, may selectively deplete CD4+ Tregs relative to effector T cells, activating effector T cells. See “Immediate versus deferred treatment for advanced prostatic cancer: initial results of the Medical Research Council Trial. The Medical Research Council Prostate Cancer Working Party Investigators Group.” Br J Urol. 79(2):235-246 (1997), which is incorporated by reference in its entirety. Other chemotherapeutic agents, such as gemcitabine, have also shown effects on the immune system, including induction of apoptosis, promotion of dendritic cell cancer antigen presentation, and facilitation of cross-priming of CD8+ T cells. See Nowak et al., “Induction of tumor cell apoptosis in vivo increases tumor antigen cross-presentation, cross-priming rather than cross-tolerizing host tumor-specific CD8 T cells.” J Immunol. 170(10):4905-4913 (2003), which is incorporated by reference in its entirety.

The combination of two or more check point inhibitors with a chemotherapeutic agent (cytostatic or cytotoxic) may benefit from targeting other non-redundant aspects of the cancer-immunity life cycle such as novel molecules, tissue site of action, immune cell population, and biological process. For example, VISTA, a molecule from the immunoglobulin superfamily (IgSF), is expressed primarily on M2 macrophages following ipilimumab (anti-CTLA-4) treatment in patients with metastatic prostate cancer. See Gao et al., “VISTA is an inhibitory immune checkpoint that is increased after ipilimumab therapy in patients with prostate cancer. Nat Med. 2017; 23(5):551-555, which is incorporated by reference in its entirety. VISTA and PD-1 have non-redundant inhibitory effects on T cells. See Liu et al., “Immune-checkpoint proteins VISTA and PD-1 non-redundantly regulate murine T-cell responses.” Proc Natl Acad Sci USA. 112(21):6682-6687 (2015), which is incorporated by reference in its entirety. As another example, gemcitabine can enhance the efficacy of a dendritic cell-based vaccine by increasing T-cell trafficking and sensitizing tumor cells to T cell-mediated lysis in a murine pancreatic cancer model. See Bauer et al., “Concomitant gemcitabine therapy negatively affects DC vaccine-induced CD8(+) T-cell and B-cell responses but improves clinical efficacy in a murine pancreatic carcinoma model.” Cancer Immunol Immunother. 63(4):321-333 (2014), which is incorporated by reference in its entirety. In a phase II clinical trial of patients with metastatic renal cell carcinoma, An additional example is the use of cyclophosphamide and multipeptide vaccine IMA901, which can improve survival in those who developed multipeptide immune responses, suggesting a diverse tumor-specific immune response generated by multiple antigens. See Walter et al., “Multipeptide immune response to cancer vaccine IMA901 after single-dose cyclophosphamide associates with longer patient survival.” Nat Med. 18(8):1254-1261 (2012), which is incorporated by reference in its entirety.

Data, however, are very limited on the efficacy of combining an immune checkpoint blockade with a low-dose chemotherapy. The combination of two or more check point inhibitors with a chemotherapeutic agent (cytostatic or cytotoxic) has been developed herein to harness additive or synergistic mechanisms of systemic cancer killing while minimizing antagonistic interactions and adverse events.

In some embodiments, the pharmaceutical compositions can further comprising a second cytotoxic or cytostatic chemotherapeutic drug. The second cytotoxic or cytostatic chemotherapeutic drug can be the same as or different from the first cytotoxic or cytostatic chemotherapeutic drug.

The immune checkpoint inhibitors are present in the pharmaceutical composition in a therapeutically effective amount. For instance, the concentration of each immune checkpoint inhibitor may range from about 0.1 to about 500 mg/ml, for instance from about 0.1 to about 300 mg/ml, from about 0.1 to about 200 mg/ml, from about 0.1 to about 100 mg/ml, from about 0.5 to about 100 mg/ml, from about 0.5 to about 50 mg/ml, from about 0.5 to about 30 mg/ml, from about 0.5 to about 20 mg/ml, from about 0.5 to about 10 mg/ml, from about 1 to about 10 mg/ml, from about 1 to about 5 mg/ml, or from about 1 to about 2 mg/ml.

The cytotoxic or cytostatic chemotherapeutic drugs are also present in the pharmaceutical composition in a therapeutically effective amount. For instance, the concentration of each cytotoxic or cytostatic chemotherapeutic drug may range from about 1 μg/ml to about 100 mg/ml, from about 1 μg/ml to about 50 mg/ml, from about 1 μg/ml to about 30 mg/ml, from about 1 μg/ml to about 20 mg/ml, from about 1 μg/ml to about 10 mg/ml, from about 1 μg/ml to about 5 mg/ml, from about 1 μg/ml to about 1 mg/ml, from about 1 to about 500 μg/ml, from about 1 to about 500 μg/ml, from about 1 to about 300 μg/ml, from about 1 to about 200 μg/ml, from about 1 to about 100 μg/ml, from about 1 to about 50 μg/ml, from about 1 to about 30 μg/ml, from about 1 to about 20 μg/ml, from about 5 to about 50 μg/ml, from about 5 to about 30 μg/ml, from about 5 to about 20 μg/ml, or from about 5 to about 10 μg/ml.

In some instances, the pharmaceutical composition comprises, consists essentially of, or consists of the CTLA-4 inhibitor at a concentration of about 0.5 to about 10 mg/ml, and the PD-1 inhibitor at a concentration of about 0.5 to about 20 mg/ml. In some instances, the pharmaceutical composition comprises the CTLA-4 inhibitor at a concentration of about 1 to about 10 mg/ml, for instance, about 2 to about 8 mg/ml, or about 5 mg/ml; and the PD-1 inhibitor at a concentration of about 1 to about 20 mg/ml, for instance, about 5 to about 15 mg/ml, or about 10 mg/ml. The cytotoxic or cytostatic chemotherapeutic drug may be present at a concentration of approximately 10 to 500 μg/ml or from about 10 to 100 mg/ml. In some instances, the pharmaceutical composition (or each component) is to be administered at a volume of about 1 ml, about 5 ml, or about 10 ml. In one embodiment, the pharmaceutical composition (or each component) is to be administered at a volume of about 1 ml.

In some instances, the composition comprises the CTLA-4 inhibitor at a concentration of about 1 to 2 mg/ml, the PD-1 inhibitor at a concentration of about 1 to 10 mg/ml and the cytotoxic or cytostatic chemotherapeutic drug at a concentration of about 250 μg/ml. For example, the composition can comprise the CTLA-4 inhibitor at a concentration of about 3.3 mg/ml, the PD-1 inhibitor at a concentration of about 6.6 mg/ml, and the cytotoxic or cytostatic chemotherapeutic drug at a concentration of approximately 16.6 μg/ml. In some instances, the composition is of a volume of at least or approximately 15 ml. In some instances, the composition is of a volume of less than approximately 15 ml.

The pharmaceutical compositions can further include one or more therapeutically effective amount of therapeutic and/or biologic agents known in the art to be effective in treating cancer, i.e., an anti-cancer agent, or a an agent known in the art to be effective in stimulating the immune system, i.e., immunostimulant or immunomodulator. Such pharmaceutical compositions can be used to treat cancer as described herein.

The pharmaceutical composition can also comprise one or more therapeutically effective amount of nucleic acid drugs. The nucleic acid drug can be, e.g., DNA, DNA plasmid, nDNA, mtDNA, gDNA, RNA, siRNA, miRNA, mRNA, piRNA, antisense RNA, snRNA, snoRNA, vRNA, etc. For example, the nucleic acid drug can be a DNA plasmid. In some instances, the DNA plasmid can comprise, consist essentially of, or consist of a nucleotide sequence encoding a gene selected from the group consisting of GM-CSF, IL-12, IL-6, IL-4, IL-12, TNF, IFNy, IFNa, and/or a combination thereof. The nucleic acid drug can have clinical usefulness, for example, in enhancing the therapeutic effects of the cells or providing a patient with a therapeutic agent. In another instance, the nucleic acid drug may function as a marker or resistance gene. The nucleotide sequence can encode a gene that can be secreted from the cells or cannot be secreted from the cells. The nucleic acid drug can encode a gene and a promoter sequence to increase expression of the gene.

One skilled in the art would appreciate that the pharmaceutical compositions can be adapted according to the individual aspects of the cancer and/or the subject, e.g., the size of the tumor, the location of the tumor, the subject, clinical evidence of drug response, etc.

The pharmaceutical composition can include a delivery agent or pharmaceutically acceptable carrier or excipient. As used herein the term “pharmaceutically acceptable carrier or excipient” includes solvents, dispersion media, coatings, antibacterial and antifungal agents, isotonic and absorption delaying agents, and the like, compatible with pharmaceutical administration. Supplementary active compounds can also be incorporated into formulation for the pharmaceutical composition that contains an antibody or antigen-binding fragment thereof as described herein.

The pharmaceutical composition containing the immune checkpoint inhibitors and the cytotoxic or cytostatic chemotherapeutic drug can be formulated for various administrative routes, including but not limited to, orally or parenterally, such as intravenously, intramuscularly, subcutaneously, intra-tumorally, intra-orbitally, intra-capsularly, intra-peritoneally, intra-rectally, intra-cisternally, intra-vasally, intra-dermally; by passive or facilitated absorption through the skin using, for example, a skin patch or transdermal iontophoresis, respectively; by being administered to the site of a pathologic condition, for example, intravenously or intra-arterially into a blood vessel supplying a tumor; or combinations thereof.

Methods of formulating suitable pharmaceutical compositions are known in the art (see, e.g., Troy, “Remington: The Science and Practice of Pharmacy” (21^{st} Ed., Lippincott Williams & Wilkins, 2006); Willig, “Drugs and the Pharmaceutical Sciences: a Series of Textbooks and Monographs” (M. Dekker, 1975); both of which are hereby incorporated by reference in their entirety. For example, solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates and agents for the adjustment of tonicity such as sodium chloride or dextrose. The pH value can be adjusted with acids or bases, such as hydrochloric acid or sodium hydroxide. The parenteral preparation can be enclosed in ampoules, disposable syringes or multiple dose vials made of glass or plastic.

The pharmaceutical composition or various components of the pharmaceutical composition (e.g., the checkpoint inhibitors, cytotoxic or cytostatic chemotherapeutic drugs, nucleic acid drugs, and/or a combination thereof) may be formulated for intra-tumorally delivery. For example, the pharmaceutical composition or various components of the pharmaceutical composition can be intra-tumorally delivered via an injection device, wherein the injection device may be part of a probe. The probes as described herein can be configured for the various ablation methods. Further, the probe can also be configured to combine the methods described herein, e.g., a cryoprobe can be configured to administer an electric pulse, a cryogen, a chemical or biological ablation agent, and/or a composition of drugs.

A combination of at least two checkpoint inhibitors and a cytotoxic or cytostatic chemotherapeutic drug administered intra-tumorally produces fewer adverse side effects and/or immune-related adverse events than a combination of the two checkpoint inhibitors (without the cytotoxic or cytostatic chemotherapeutic drug) administered intravenously. The combination of these three or more immune-stimulating drugs delivered intra-tumorally may be sufficient to trigger a systemic CD4+ and CD8+ T-cell mediated anti-tumor immune response which can eradicate distant metastatic tumor sites, including in the central nervous system in mice. This local combination strategy may also generate a better CD8+ memory anti-tumor immune response because it prevents late tumor relapses as opposed to systemic delivery of antibodies.

The combination of at least two checkpoint inhibitors and a cytotoxic or cytostatic chemotherapeutic drug is superior to a combination of at least two checkpoint inhibitors (but without a cytotoxic or cytostatic chemotherapeutic drug) due to the additive effect on the ability of these immune-stimulating drugs to deplete intra-tumoral regulatory T Cells (Tregs). Additionally, generation of an efficient systemic adaptive anti-cancer immune response can be optimized by intra-tumoral immunization strategies that combine Treg depletion with immunogenic tumor cell death and activation of dendritic cells.

Traditionally, checkpoint inhibitors are administered intravenously, which can result in serious and sometimes fatal systemic toxicities as a result of non-specific distribution of these cytocidal agents in the body. The non-specific distribution of these agents kills both cancer cells and normal cells and can negatively impact the treatment regimen and patient outcome. The intra-tumoral methods can reduce systemic toxicity and produce fewer side effects by sequestering the drugs in the tumor microenvironment and sparing normal cells and tissues from the toxicity of the drugs (see Marabelle et al., “Intratumoral Immunization: A New Paradigm for Cancer Therapy” Clin. Cancer Res. 20(7): 1747-56 (2014), which is incorporated herein by reference in its entirety). The intra-tumoral injection of immune stimulating drugs can reduce systemic toxicity and product fewer side effects by preventing their circulation at high concentrations in the blood. This route of delivery also produces much higher concentrations of immunostimulatory products in the cancer micro-environment than with systemic infusion, thereby potentiating better efficacy. On the other hand, this route of delivery also allows for lowering the amount of the administered compositions necessary to be therapeutically effective.

Multiple costimulatory and cohibitory receptors influence control T-cell activation, proliferation, and gain or loss of effector function, including CTLA-4. CTLA4 binds B7-1 and B7-2 ligands, promoting anti-cancer activity by activating CD8+ cytotoxic T cells and concomitantly depleting CD4+ Tregs (see Selby et al., “Anti-CTLA-4 antibodies of IgG2a isotype enhance antitumor activity through reduction of intratumoral regulatory T cells” Cancer Immunol. Res. 1:32-42 (2013), which is incorporated herein by reference in its entirety). These results may explain the systemic anti-cancer immune response generated in mouse models with local low dose delivery of anti-CTLA-4. Low doses of anti-CTLA-4 antibody injected around an established mouse colon carcinoma were able to eradicate the local tumor and prevent development of cancer at a distant non-injected site (abscopal effect) by direct enhancement of cancer-specific CD8+ T-cell responses (see Fransen et al., “Controlled local deliver of CTLA-4 blocking antibody induces CD8+ T-cell-dependent tumor eradication and decreases risk of toxic side effects” Clin. Cancer Res. 19:5381-9 (2013), which is incorporated herein by reference in its entirety).

Moreover, by combining techniques that target both the cancer cells and the immune system, the pharmaceutical composition can be more effective at not only inhibiting the cancer but also triggering an effective antitumor immune response. This antitumor immune response may then target metastatic sites and eliminate cancer throughout the subject.

Pharmaceutical compositions suitable for injection can include sterile aqueous solutions (where water soluble), dispersions, and sterile powders for the extemporaneous preparation of sterile injectable solutions or dispersion. For intravenous administration, suitable carriers include physiological saline, bacteriostatic water, Cremophor EL™ (BASF, Parsippany, N.J.), or phosphate buffered saline (PBS). It is desirable that the composition be sterile and fluid to the extent that easy syringability exists. The pharmaceutical composition should be stable under the conditions of manufacture and storage and be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants.

Prevention of the action of microorganisms can be achieved by various antibacterial and antifungal agents, for example, parabens, chlorobutanol, phenol, ascorbic acid, thimerosal, and the like. In many cases, it is desirable to include isotonic agents, for example, sugars, polyalcohols such as mannitol, sorbitol, and sodium chloride in the pharmaceutical composition. Prolonged absorption of the injectable compositions can be brought about by including in the pharmaceutical composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the desirable methods of preparation are vacuum drying and freeze-drying, which yield a powder of the active ingredient plus any additional desired ingredient from a previously sterile-filtered solution thereof. In some embodiments, the pharmaceutical compositions can be prepared with carriers that will protect the active compounds against rapid elimination from the body, such as a controlled release formulation, including implants and microencapsulated delivery systems.

The pharmaceutical compositions can be included in a container, pack, cartridge, or dispenser together with instructions for administration.

The term “administer” or “administration” in relation to the methods include not only the actions of prescriptions and/or instructions from a medical professional, but also the actions of taking the prescriptions and/or instructions of a patient and the actions of actually taking the composition or treatment steps by the patient.

Another aspect of the invention provides methods of treating a tumor or a cancer in a patient. The method can comprise, consist essentially of, or consist of administering to the patient in need a composition comprising at least two immune checkpoint inhibitors and at least one cytotoxic or cytostatic chemotherapeutic drug, each being present in the composition in a therapeutically effective amount to treat the tumor or cancer. The composition can optionally contain a pharmaceutically acceptable carrier. For example, the administered composition may be the pharmaceutical compositions described herein.

All above embodiments relating to the aspect of the pharmaceutical composition, including suitable immune checkpoint inhibitors, suitable cytotoxic or cytostatic chemotherapeutic drug, suitable optional pharmaceutically acceptable carriers, their effective amounts for treating tumor or cancer, and the formulations of the pharmaceutical composition for various administrative routes are applicable in this aspect of the method of treating a tumor or a cancer in a patient

In some instances, the method comprises, consists essentially of, or consists of administering the composition to the patient intratumorally.

In some embodiments, the method comprises, consists essentially of, or consists of administering to the patient a composition comprising at least two different immune checkpoint inhibitors, each being an inhibitor of an immune checkpoint molecule selected from the group consisting of CD137, CD134, PD-1, KIR, LAG-3, PD-L1, PDL2, CTLA-4, B7.1, B7.2, B7-DC, B7-H1, B7-H2, B7-H3, B7-H4, B7-H5, B7-H6, B7-H7, BTLA, LIGHT, HVEM, GALS, TIM-3, TIGHT, VISTA, 2B4, CGEN-15049, CHK 1, CHK2, A2aR, TGF-β, PI3Kγ, GITR, ICOS, DO, TLR, IL-2R, IL-10, PVRIG, CCRY, OX-40, CD160, CD20, CD52, CD47, CD73, CD27-CD70, and/or CD40; and at least one cytotoxic or cytostatic chemotherapeutic drug, in an amount effective to treat the tumor or cancer. In some embodiments, the at least two checkpoint inhibitors comprises a CTLA-4 inhibitor, a PD-1 inhibitor. In some embodiments, the at least two checkpoint inhibitors comprises a CTLA-4 inhibitor and a PD-L1 inhibitor.

In some embodiments, the method comprises, consists essentially of, or consists of administering to the patient a composition comprising at least two immune checkpoint inhibitors and at least one cytotoxic or cytostatic chemotherapeutic drug selected from the group consisting of asparaginase, bleomycin, busulphan, carboplatin, cetuximab, cisplatin, cyclophosphamide, BCG, chloramphenicol, colchicine, cyclosporin, dacarbazine, doxorubicin, etoposide, fludarabine, gemcitabine, ifosfamide, irinotecan, lomustin, melphalan, methotrexate, mitomycin, mitoxantrone, paclitaxel, procarbazine, rituximab, temozolomide, thitepa, vinblastine, vincristine, zidovudine, and combinations thereof, in an amount sufficient to treat the tumor or cancer.

In some instances, the method further comprises administering a therapeutically effective amount of a nucleic acid drug to the tumor or cancer. Administering the combination of at least two checkpoint inhibitors and at least one cytotoxic or cytostatic chemotherapeutic drug produces fewer side effects and/or immune-related adverse events than administering the combination of the checkpoint inhibitors (e.g., without a cytotoxic or cytostatic chemotherapeutic drug).

As discussed above, the administration of the composition or its components can be conducted via various routes, including but not limited to, administering orally or parenterally, such as intravenously, intramuscularly, subcutaneously, intra-tumorally, intra-orbitally, intra-capsularly, intra-peritoneally, intra-rectally, intra-cisternally, intra-vasally, intra-dermally; administering by passive or facilitated absorption through the skin using, for example, a skin patch or transdermal iontophoresis, respectively; administering to the site of a pathologic condition, for example, intravenously or intra-arterially into a blood vessel supplying a tumor; or combinations thereof.

The pharmaceutical composition or its components can be administered in an effective amount, at dosages, and for periods of time necessary to achieve the desired result. An effective amount can be administered in one or more administrations, applications or dosages. A therapeutically effective amount of a pharmaceutical composition (i.e., an effective dosage) depends on the pharmaceutical composition selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the pharmaceutical compositions described herein can include a single treatment or a series of treatments.

In some instances, the composition is administered to the patient's tumor or cancer using an injection device. The injection device may comprise multiple tines or a single tine. The compositions can be administered using a probe (that serves different purposes) as described herein.

In some embodiments, the compositions described herein can be administered in one or more administrations. These one or more administrations can be of the same or different methods of administration as described herein, for example, subcutaneously, intravenously, intramuscularly, intra-tumorally or any combinations thereof.

In some embodiments, a first composition is administered intra-tumorally and a second composition is administered subcutaneously. In some embodiments, a first and second compositions are administered simultaneously, in sequence, or in a series of treatments. In some embodiments, a first and the second compositions are the same, different, or the same in part. In some embodiments, the treatment methods include two or more administrations.

In some embodiments, a first administration is an intra-tumoral administration of at least two checkpoint inhibitors (e.g., a PD-1 inhibitor and a CTLA-4 inhibitor) and at least one cytotoxic or cytostatic chemotherapeutic drug.

Dosage regimens can be adjusted to provide the optimum therapeutic response. For example, several divided doses can be administered daily or the dose can be proportionally reduced as indicated by the exigencies of the therapeutic situation. Those skilled in the art will be aware of dosages and dosing regimens suitable for administration of the new monoclonal antibodies disclosed herein or antigen-binding fragments thereof to a subject. See e.g., Physicians' Desk Reference 2008 (62^{nd} Ed., Thomson Reuters, 2008), which is incorporated herein by reference in its entirety. For example, dosage, toxicity, and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the treatment method, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The composition can be administered in a single dose or can be administered in more than one dose. As discussed above, the dosage of the immune checkpoint inhibitors, when measured by the concentration in the pharmaceutical composition, may range from about 0.1 to about 500 mg/ml, for instance from about 0.1 to about 300 mg/ml, from about 0.1 to about 200 mg/ml, from about 0.1 to about 100 mg/ml, from about 0.5 to about 100 mg/ml, from about 0.5 to about 50 mg/ml, from about 0.5 to about 30 mg/ml, from about 0.5 to about 20 mg/ml, from about 0.5 to about 10 mg/ml, from about 1 to about 10 mg/ml, from about 1 to about 5 mg/ml, or from about 1 to about 2 mg/ml. The dosage of the cytotoxic or cytostatic chemotherapeutic drugs, when measured by the concentration in the pharmaceutical composition, may range from about 1 μg/ml to about 100 mg/ml, from about 1 μg/ml to about 50 mg/ml, from about 1 μg/ml to about 30 mg/ml, from about 1 μg/ml to about 20 mg/ml, from about 1 μg/ml to about 10 mg/ml, from about 1 μg/ml to about 5 mg/ml, from about 1 μg/ml to about 1 mg/ml, from about 1 to about 500 μg/ml, from about 1 to about 500 μg/ml, from about 1 to about 300 μg/ml, from about 1 to about 200 μg/ml, from about 1 to about 100 μg/ml, from about 1 to about 50 μg/ml, from about 1 to about 30 μg/ml, from about 1 to about 20 μg/ml, from about 5 to about 50 μg/ml, from about 5 to about 30 μg/ml, from about 5 to about 20 μg/ml, or from about 5 to about 10 μg/ml.

In some embodiments, the composition is administered in a volume of less than about 15 ml. In some embodiments, the composition is administered in a volume of about 15 ml.

In some embodiments, the composition is administered in a volume of no more than about 15 ml, no more than about 10 ml, no more than about 5 ml, or no more than about 1 ml. In some embodiments, the composition (or each component) is administered in a volume of about 1 ml, about 5 ml, or about 10 ml.

In some embodiments, the dosage of the immune checkpoint inhibitors, when measured based on the weight of the subject, can range from about 0.01 to about 10 mg/kg, for instance, from about 0.05 to about 10 mg/kg, from about 0.1 to about 10 mg/kg, from about 0.1 to about 5 mg/kg, from about 0.1 to about 3 mg/kg, from about 0.1 to about 2 mg/kg, from about 0.1 to about 1 mg/kg, or from about 0.5 to about 1 mg/kg.

In some embodiments, the dosage of the cytotoxic or cytostatic chemotherapeutic drugs, when measured based on the weight of the subject, can range from about 1 μg/kg to about 10 mg/kg, for instance, from about 1 μg/kg to about 10 mg/kg, from about 2 μg/kg to about 10 mg/kg, from about 2 μg/kg to about 5 mg/kg, from about 2 μg/kg to about 3 mg/kg, from about 2 μg/kg to about 2 mg/kg, from about 2 μg/kg to about 1 mg/kg, from about 2 to about 500 μg/kg, from about 2 to about 100 μg/kg, from about 2 to about 50 μg/kg, or from about 2 to about 10 μg/kg.

In some instances, the cytotoxic or cytostatic chemotherapeutic drug may be administered at a dosage ranging from about 0.1 to about 1000 mg/m², for instance, from about 10 to about 600 mg/m². In one embodiment, the cytotoxic or cytostatic chemotherapeutic drug is administered in a low dose, for instance less than about 500 mg/m², less than about 400 mg/m², or less than about 300 mg/m².

In one embodiment, the dose of the cytotoxic or cytostatic chemotherapeutic drug in each administration is about 0.25% to about 75% of its maximum tolerated dose following a traditional dosing regimen. For instance, the cytotoxic or cytostatic chemotherapeutic drug is administered in a low dosage that, the dose per administration is about 1%, about 5%, about 10%, about 15%, about 20%, about 25%, about 30%, about 35%, about 40%, about 45%, about 50%, about 55%, about 60%, about 65%, about 70%, or about 75%, of the maximum tolerated dose.

In some embodiments, the intratumoral administration of the pharmaceutical composition described herein produces fewer adverse side effects and/or immune-related adverse events, when compared to the conventional IV administration of the same composition. Adverse side effects and immune-related adverse events of conventional IV administration include gastrointestinal, respiratory, neurologic, endocrine, dermatologic, fatigue, renal, and hepatic effects.

In some embodiments, the administration of the pharmaceutical composition described herein (i.e., comprising at least two immune checkpoint inhibitors and at least one cytotoxic or cytostatic chemotherapeutic drugs) produces fewer adverse side effects and/or immune-related adverse events in vivo, when compared to the administration of a same pharmaceutical composition comprising the at least two immune checkpoint inhibitors and without the cytotoxic or cytostatic chemotherapeutic drugs.

In some instances, the method of treating a tumor or cancer comprises, consists essentially of, or consists of ablating at least a portion of the tumor or cancer.

Combining the pharmaceutical composition containing at least two checkpoint inhibitors and a cytotoxic or cytostatic chemotherapeutic drug with the ablation method may provide a systemic, durable, and reproducible cancer immunity.

Ablative techniques, such as cryotherapy and radiation therapy, when used in isolation, produce regulatory T cell inhibition, effector T and B cell activation, and cancer-associated antigen release (see Maia et al., “A comprehensive review of immunotherapies in prostate cancer.” Crit Rev Oncol Hematol. 113:292-303 (2017), which is incorporated herein by reference in its entirety), effectively creating an adjuvant effect that stimulates the cytotoxic T lymphocyte response. For example, cells rendered necrotic by freeze-thawing have immunostimulatory activity when injected in vivo as they enhance T cell responses to co-injected antigens. See Shi et al., “Cell injury releases endogenous adjuvants that stimulate cytotoxic T cell responses.” Proc Natl Acad Sci USA. 97(26):14590-14595 (2000), which is incorporated herein by reference in its entirety.

The combination of immunotherapy and ablation therapy can enhance the immune response, perhaps by exploiting the benefits of different mechanisms of action. In the 3LL murine Lewis lung carcinoma model, cryotherapy combined with immunotherapy can cause robust and tumor-specific CTL responses, increase Th1 responses, significantly prolong survival, and significantly reduce the incidence of metastases. See Machlenkin et al., “Combined dendritic cell cryotherapy of tumor induces systemic antimetastatic immunity.” Clin Cancer Res. 11(13):4955-4961 (2005), which is incorporated herein by reference in its entirety.

Similar treatments can protect mice that have survived primary ovalbumin-transfected B16 melanoma from re-challenge with parental tumor. Cryoablation combined with CTLA-4 blockade or regulatory T-cell depletion may also protect mice from outgrowth of cancer challenges and lead to in vivo enhancement of cancer-specific T-cell numbers. See den Brok et al., “Synergy between in situ cryoablation and TLR9 stimulation results in a highly effective in vivo dendritic cell vaccine.” Cancer Res. 66(14):7285-7292 (2006), which is incorporated herein by reference in its entirety. In the TRAMP C2 mouse model of prostate cancer, cryoablation and CTLA-4 blockade of primary cancer may prevent outgrowth of secondary cancers that were seeded by challenge at a distant site. See Waitz et al., “Potent induction of tumor immunity by combining tumor cryoablation with anti-CTLA-4 therapy.” Cancer Res. 72(2):430-439 (2012), which is incorporated herein by reference in its entirety. Although growth of secondary tumors may not be unaffected by cryoablation alone, the combination treatment can be sufficient to slow growth or trigger rejection. In addition, secondary tumors are highly infiltrated by CD4+ T-cells and CD8+ T-cells and there is a significant increase in the ratio of intratumoral T effector cells to CD4+FoxP3+ T regulatory cells compared with monotherapy. Accordingly, cryoimmunotherapy may be able to modulate intratumoral accumulation and systemic expansion of CD8+ T cells specific for the TRAMP C2-specific antigen SPAS-1.

The combination of radiation therapy and CTLA-4 blockade can also induce a CD8+ T-cell mediated antitumor response capable of inhibiting metastases outside the field of radiation and extending the survival of the mice, a response was not observed with CTLA-4 blockade alone. See Demaria et al., “Immune-mediated inhibition of metastases after treatment with local radiation and CTLA-4 blockade in a mouse model of breast cancer.” Clinical Cancer Research. 11(2):728-734 (2005), which is incorporated herein by reference in its entirety.

Radiation therapy followed by PD-1 blockade or CTLA-4 blockade may bring additive benefit through non-redundant mechanisms or synergistic effects. See Twyman-Saint Victor et al., “Radiation and dual checkpoint blockade activate non-redundant immune mechanisms in cancer.” Nature. 520(7547):373-377 (2015); Dovedi et al., “Acquired resistance to fractionated radiotherapy can be overcome by concurrent PD-L1 blockade.” Cancer Res. 74(19):5458-5468 (2014); Golden et al., “An abscopal response to radiation and ipilimumab in a patient with metastatic non-small cell lung cancer.” Cancer Immunol Res. 1(6):365-372 (2013), which are incorporated herein by reference in their entirety.

The method of ablation described herein influences at least two factors that are known to influence the immunologic response to an ablated tumor. One is the effect of the ablation process on the protein structure and therefore the antigenicity of the tumor proteins. The second factor is the mechanism of cell death related to the ablation modality.

Necrosis (immediate cell death), under certain conditions, ruptures the cell membrane and causes cell membrane fragments and a wide range of intracellular contents to spill out of the devitalized cells into the extracellular environment that causes co-stimulation of dendritic cells, leading to T Cell proliferation and activation. In contrast, apoptosis (programmed cell death), another form of irreversible injury, in which cells shrivel up and die over time, usually within a few days. Apoptosis leaves the cells intact, confines the cellular contents, and prevents co-stimulation. This lack of intracellular exposure and co-stimulation mutes the immunologic effect by preventing T cell activation and proliferation. Therefore, necrosis optimizes immunogenic stimulation, whereas apoptosis usually elicits little or no immune response.

Ablation does not remove the treated tissue, unlike surgical extirpation; instead, the altered cell mass persists in situ, with subsequent removal or sequestration by the body's defense and healing mechanisms. Therefore, one of the unique aspects of ablation, versus surgical removal, is that the tumor is left in situ for the body's defense and healing mechanisms to remove it. This creates an opportunity to harness the body's immune defense mechanisms to recognize the dead tumor and essentially auto-immunize the patient against potential cancer neo-antigens (i.e., against patient's own cancer) (see Veenstra et al., “In situ immunization via non-surgical ablation to prevent local and distant tumor recurrence” Oncoimmunology 4(3): e989762 (2015), which is incorporated herein by reference in its entirety). Moreover, by stimulating the immune system to the cancer cell antigens, the methods disclosed herein can (i) treat primary tumors; (ii) activate the immune response to cancer cell antigens; and (iii) induce immune system targeting of metastatic lesions.

The ablating step can be performed, e.g., prior to, concurrently with, and/or after the administration of the compositions as described herein.

The ablating step can be performed by using various ablation methods or combinations thereof known in the art. Suitable ablation methods include cold ablation, such as cryoablation; thermal ablation, such as radio frequency (RF) ablation, microwave ablation, laser, photo, or plasma ablation, ultrasonic ablation, high-intensity focused ultrasound (HIFU) ablation, or steam ablation; electrical ablation, such as reversible electroporation (RE), irreversible electroporation (IRE), radiofrequency electrical membrane breakdown (RF-EMB), RF-EMB type ablation, ablation with ultra-short electrical pulse; ablation using photodynamic therapy; mechanical or physical ablation such as ablation using non-thermal shock waves, cavitation, or other mechanical physical means to create cell disruption; chemical ablation, such as ablation by injection of chemicals, e.g., alcohol, hypertonic saline, acetic acid, etc.; ablation with biologics, such as oncolytic viruses; or any combination thereof.

These different types of ablation methods can have different outcomes on the protein structures and mechanism of cell death. For example, heat ablation destroys structures due to denaturing proteins and it also destroys the underlying collagen matrix of the tissue. This disruption of the proteins and tissue makes a robust immunologic response unlikely. Cold ablation, e.g. cryoablation, can denature proteins and can disrupt both protein and tissue structure. Irreversible electroporation (IRE) and non-thermal ablation modalities, e.g., RF-EMB, are structure sparing and can therefore be used to treat cancers in the pancreas, central liver, and other areas such as the head and neck. IRE is a technique where an electrical field is applied to cells to increase the permeability of the cell membrane. The high voltage of IRE destroys the target cells while leaving neighboring cells unaffected. Radiofrequency electrical membrane breakdown (RF-EMB) is another non-thermal modality that produces necrosis by complete breakdown of the cell membrane electrically (see WO 2015/085162, which is incorporated herein by reference in its entirety). Under certain conditions, RF-EMB can also be used to deliver DNA plasmids. Reversible electroporation (RE) can also be used to deliver DNA plasmids. RE is similar to IRE, however the electricity applied to the target cells is below the electric field threshold of the target cells. Therefore, the cells can recover when the electric field is removed and rebuild their cellular membranes and continue with cellular functions. RE can be used as a tool for gene therapy as the reversible element allows for entry of nucleic acids (e.g. DNA plasmids) into a viable cell. Exemplary ablation methods and brief descriptions of their mechanism are summarized in Table 1.

TABLE 1
Exemplary Ablation Methods.

| METHOD | MECHANISM | DESCRIPTION |
|---|---|---|
| Thermal | | |
| Microwave | Heat and | Creates coagulation necrosis with |
| | mechanical | friction and heat |
| HIFU | Heat | Creates necrosis by focusing energy |
| | | into a small area creating heat |
| Laser | Heat | Creates necrosis with light energy |
| RF Thermal | Heat and | Creates cellular desiccation and |
| | mechanical | protein coagulation |
| Steam | Heat | Creates coagulation necrosis with |
| | | heat |
| Cryosurgery | Cold | Creates necrosis by dehydration and |
| | | ice formation |
| Non-Thermal | | |
| Alcohol, | Chemical | Creates coagulative necrosis via |
| Hypertonic | | dehydration and protein coagulation |
| Saline, Acetic | | |
| Acid Injections | | |
| Photodynamic | Chemical | Creates cell damage by reactive |
| | | oxygen species and destroying |
| | | vessels |
| IRE and N- | Electrical | Creates apoptosis with preservation |
| TIRE (Nanoknife) | | of vessels; delayed necrosis |

Any ablation method described herein can be used alone or in combination with one or more other ablation methods. Two or more ablation methods may be applied sequentially or concurrently. In some cases, a combination of ablation methods may have a synergistic effect on the tissue. A non-limiting list of combinations includes, for example, heat ablation and RF-EMB, cryoablation and RF-EMB, IRE and RF-EMB, RE and RF-EMB, IRE and cryoablation, heat ablation and cryoablation, heat ablation and IRE, RE and IRE, heat ablation with RE, and any combination in which two or more methods are used.

In some cases, methods described herein create an RF-EMB type lesion using a combination of RF-EMB and cryoablation techniques. This combination of ablation methods can produce a synergistic effect on the tissue. The synergistic effect can be the creation of an RF-EMB type lesion with less required energy input than with other means. The result, for instance in liver tissue includes: in areas adjacent to aseptic non-inflammatory coagulative necrosis, there is alteration of liver architecture, including dilation of bile duct canaliculi, as well as unique diffuse alteration of cytoplasmic organelles, including distortion of mitochondrial cristae and vacuolization of endoplasmic reticulum.

One of skill in the art would appreciate that the administration method described herein can be adapted according to the individual aspects of the cancer, e.g., the size of the tumor, the location of the tumor, the subject, etc. One of skill in the art would appreciate that the variables of each of the various ablation methods are known and described in the art (including, for example, Percutaneous Prostate Cryoablation (Edited by Onik, Rubinsky, Watson, and Ablin. Quality Medical Publishing, St Louis, Mo., 1995), which is incorporated herein by reference in its entirety).

As examples of the variability and variety of ablation parameters, the process of cryoablation includes adjustable variables such as the number of freeze-thaw cycles, the speed of the freeze, the thaw portion of the cycle, to influence the outcome of the ablation, e.g., the size of the lesion, the damage to the surrounding tissue, and the immune response to the lesion. Similarly, the process of RF-EMB includes adjustable variables such as the strength of the electric field, frequency, polarity, shape duration, number and spacing, etc., which can similarly influence the outcome of the ablation. The proximity of a tumor cell to the electric pulse will determine the strength and outcome of the RF-EMB on any particular cell. For example, as the electric field strength diminishes from the point of administration (e.g., the probe), the cells furthest from the point of administration are treated with a lower strength electric field and as such may not be ablated but rather reversibly electroporated.

In some instances, a first portion or all of a tumor is ablated using a first ablation method and a second portion or all of the tumor is ablated using a second ablation method. The first and the second ablation methods can be the same or different. The first and the second portions of the tumor or cancer can be the same or different portions of the tumor or cancer. In some instances, the ablating is performed prior to administration of the composition. In some cases, ablating is performed concurrently with administration of the composition or performed after administration of the composition. In some cases, ablating is performed concurrently to and after administration of the composition.

In some embodiments, the ablating of at least a portion of the tumor or cancer is performed using both RF-EMB and cryoablation.

In some instances, the ablating step is, at least in part, performed using cryoablation. As discussed above, cryoablation is a process that uses cold to destroy tissue and creates necrosis by dehydration and ice formation. Cryoablation technique typically involves inserting a hollow needle (cryoprobe) into a tissue and then supplying a cryogen to the tip of the cryoprobe. The cryoablation can be performed using more than one cryoprobe. The cryoablation can also be performed using any of the multi-purpose probes described herein.

The tissue temperature is decreased to a temperature that correlates with the complete coagulation necrosis. Common cryoablation techniques involve the use of high pressure (e.g., about 80 psi) liquid nitrogen systems or high pressure (e.g., 3000-4500 psi) argon gas systems. Usually, the freezing of the tissue is subsequently followed by its thawing (usually using a helium gas or resistive heating), which leads to the disruption of cell membranes and induces cell destruction. The cell destruction is further accelerated upon the repetition of the freeze-thaw cycles. In some instances, the cryoablation step can comprise, consist essentially of, or consist of at least 1 freeze-thaw cycle. For example, the cryoablation can comprise between 1 and 4 freeze-thaw cycles. The freeze portion of the freeze-thaw cycle can be, e.g., at least or about 30 seconds long. The freeze portion of the freeze-thaw cycle can range from about 30 seconds to about 15 minutes, from about 30 seconds to about 12 minutes, from about 30 seconds to about 10 minutes, or from 30 seconds to about 5 minutes. The thawing time can be at least or about 30 seconds long. For instance, the thawing time can range from about 30 seconds to about 15 minutes, from about 30 seconds to about 12 minutes, from about 30 seconds to about 10 minutes, or from 30 seconds to about 5 minutes. In some embodiment, the entire cryoablation step lasts for no more than 30 minutes, no more than 25 minutes, no more than 20 minutes, no more than 15 minutes, no more than 10 minutes, or no more than 5 minutes.

As discussed above, one benefit of the treatment method provided herein is inducing immune-stimulating necrosis by minimally-invasive ablation. In some embodiments, a minimally-invasive ablation is carried out by insertion of a single probe (e.g., a cryosurgery needle probe); the ablating treatment step lasts for no more than 5 minutes to achieve the desired temperature and effect.

Another benefit of the treatment method provided herein is safeguarding adjacent structures by limiting the size of the ablation. Desirably, the size of the ablation is no more than 1 cm³ in diameter, thereby destroying about 10⁸ cancer cells (see Del Monte, “Does the cell number 10(9) still really fit one gram of tumor tissue? Cell Cycle 8:505-6 (2009), which is incorporated herein by reference in its entirety). A circumferential 1 mm-wide rim of cell injury can separate the central core of dead cancer cells from the surrounding intact unaffected cells. Safeguarding adjacent structures such as blood vessels and lymphatic channels at the edge of treated cancer can facilitate inflow and egress of immune cells. In some embodiments, a minimally-invasive ablation is carried out by insertion of a single probe (e.g., a cryosurgery needle probe) with a diameter of no more than 2 mm, for example, no more than 1.5 mm, or no more than 1 mm.

The freeze portion of the freeze-thaw cycle can be performed, e.g., at a temperature between about −30° C. and about −196° C., for instance, from about −30 to about −80° C., from about −35 to about −45° C., from about −35 to about −40° C., from about −40 to about −50° C., from about −40 to about −45° C., or at about −40° C.

As discussed above, one benefit of the treatment method provided herein is preserving cancer neo-antigens by employing minimal thermal ablation. Cancer neo-antigens are unique foreign proteins present on the internal and external surfaces of cell membranes. These neo-antigens are immunodeterminants and may be critical in immunotherapy treatment for early cancer recognition and destruction by antigen-specific T-cells. See Desrichard et al., “Cancer neoantigens and applications for immunotherapy” Clin. Cancer Res. 22: 807-12 (2016), which is incorporated herein by reference in its entirety. Preservation of neo-antigens is required for immune activation. The immune system is capable of controlling cancer development and mediating regression by generating and activating of cancer-neo-antigen-specific dendritic cells and cytotoxic CD8+ T-cells. This allows the immune cells to recognize and target neoantigens on cancer cells at metastatic sites such as lymph nodes and bone.

Most cancer ablation methods induce necrosis but many fail to preserve the 3-dimensional protein structure of cancer neo-antigens (see Onik et al., “Electrical membrane breakdown (EMB): Preliminary findings of a new method of non-thermal tissue ablation” J. Clin. Exp. Pathol. 7:5-11 (2017), which is incorporated herein by reference in its entirety). This can be undesirable as it prevents neo-antigen identification by immune cells.

Accordingly, in some embodiments, cryosurgery is employed at relatively low temperatures of about −40° C., rather than the usual −80° C., to preserve the 3-dimensional structure of the neo-antigens. Cryoablation at about −40° C. creates immune-stimulating necrosis by exceeding the threshold of cell death, while avoiding or minimizing thermal destruction of the protein neo-antigen destruction. See Larson et al., “In vivo interstital temperature mapping of the human prostate during cryosurgery with correlation to histopathologic outcomes” Urology 55:547-52 (2000), which is incorporated herein by reference in its entirety.

The thaw portion of the freeze-thaw cycle can be an active thaw process, i.e., with the addition of heat, and/or a passive thaw process, i.e., without the addition of heat.

In some instances, the methods further comprise, consist essentially of, or consist of administering a series of electrical pulses, thereby reversibly electroporating the cells adjacent to the ablation site. In some instances, the administration of the electrical pulses is performed concurrently with the ablation. In some instances, the administration of electrical pulses is performed before the ablation. In some instances, the administration of electrical pulses is performed after the ablation. The electrical pulses can be administered via the cryoprobe. In some instances, the series of electrical pulses comprise approximately 1 to 1000 pulses and/or comprise a frequency between 100 and 500 kHz. In some instances, the series of electrical pulses comprise approximately 1 to 4000 pulses and/or comprise a frequency between 100 and 500 kHz. In some instances, the series of electrical pulses comprise approximately 1 to 4000 pulses. In some cases, the series of electrical pulses comprises a frequency between 100 and 500 kHz. The electrical pulses can be, e.g., bipolar and/or have instant charge reversal.

In some instances, the methods further comprise, consist essentially of, or consist of administering a therapeutically effective amount of a nucleic acid drug to the tumor or cancer. In some instances, the methods further comprise, consist essentially of, or consist of administering a therapeutically effective amount of a nucleic acid drug to the ablation site. The nucleic acid drug can be any of the therapeutic nucleic acids described herein. The nucleic acid may be administered via any of the methods for administering the pharmaceutical composition described herein. For instance, the nucleic acid may be delivered with using reversible electroporation (RE), which can be modified to determine the range, reversibility and delivery of the electroporation around the ablation site. The variables of electroporation are known in the art (see Kee et al., Clinical aspects of electroporation (Springer, New York, 2011), which is incorporated herein in its entirety). The nucleic acid drug can be administered before or during the process of electroporation.

The administration of the nucleic acid drug can be performed prior to, concurrently with, and/or after the administration of the composition containing the immune checkpoint inhibitors and cytotoxic or cytostatic chemotherapeutic drug, as described herein. The administration of the nucleic acid drug can be performed prior to, concurrently with, and/or after the ablation step. When the electrical pulses are applied, the administration of the nucleic acid drug can be performed before, concurrently with, and/or after the administration of the electric pulses.

In some instances, the nucleic acid drug is a DNA plasmid. For example, the DNA plasmid can comprise a nucleotide sequence encoding a gene selected from the group consisting of GM-CSF, IL-12, IL-6, IL-4, IL-12, TNF, IFNy, IFNa, and/or a combination thereof.

Ablating of at least a portion may be performed using RF-EMB, e.g., using a probe. The probe can be any of the probes disclosed herein. In some instances, the probe administers a series of electrical pulses, thereby creating an ablation site immediately adjacent or in relation to the probe and reversibly electroporating the cells adjacent or in relation to the ablation site.

In some instances, the series of electrical pulses comprise approximately 1 to 1000 pulses. In some instances, the series of electrical pulses comprises approximately 1 to 4000 pulses. In some instances, the electrical pulses comprise a frequency between 100 and 500 kHz. The electrical pulses can be bipolar. The electrical pulses can also have an instant charge reversal.

In some instances, certain ablation method can create an unique tissue necrosis characterized by the destruction of cell membrane, including many thermal ablations (e.g., cryoablation) and FR-EMB. Upon destruction of the cellular membrane, the intracellular components and constituent parts of the cell membrane disperse into the extracellular space whereby immunologic identification and response is enhanced. For instance, imaging of a lesion created by RF-EMB ablation on liver tissue shows a unique form of cellular damage with disruption of the cellular membrane and loss of internal organelles such as mitochondria. This is different than other types of ablation methods (for example, IRE) which create tissue apoptosis, in which the cell membrane remains intact, the cells dies an apoptotic death, and the cell does not expose cellular antigens. In some cases, the degree of cell membrane destruction decreases as distance from the point of ablation increases.

As used herein, the term “RF-EMB type ablation” refers to any ablation technique or combination of techniques which, when performed, yields essentially the same results as RF-EMB ablation. As described herein, RF-EMB ablation and RF-EMB type ablation form lesions having any one or more of the following characteristics: destroyed cellular membranes, non-denatured cellular proteins, non-denatured membrane antigens, enhanced antigen presentation, being capable of co-stimulating the immune system, and the immediate surroundings of the lesion being able to conduct immunologic capable cells and signaling molecules.

In some instances, the portion of the tumor that is ablated comprises cancer cells, and the ablating is performed under conditions that disrupt cellular membranes of the cells and expose the intracellular components and membrane antigens of the cells, e.g., to the body's immune system.

The ablating step can be carried out by cryoablation in a minimally invasive manner. For instance, the cryoablation can be performed, e.g., by using a single probe, with total ablating time of no more than 5 minutes, using a single probe with a diameter of no more than 1 mm, and/or at a temperature from about −35 to about −45° C.

Such minimally invasive ablation brings at least one of the following benefits: intracellular components and membrane antigens of the cells are not or minimally denatured by the ablation; the immediate surroundings of the ablated portion of the tumor are capable of conducting immunologic capable cells and signaling molecules into and out of the ablated tissue; the amount of exposed intracellular components and membrane antigens of the cells is sufficient to stimulate the immune system; and/or the amount of exposed intracellular components and membrane antigens of the cells do not or minimally create immune tolerance. In one embodiment, the minimally invasive ablation preserves the structure of cancer neo-antigen such that the antigen stimulates the immune system.

In some instances, the step of administering the composition and the ablating step are carried out using a same device that comprise an ablation module and an injection module (e.g., an ablation probe that comprises an injection device). In some examples, the ablation probe can further comprise a pump for controlling the speed at which the composition is administered.

In some embodiments, the composition is administered using a device different from the device used for the ablating step.

In some instances, the method further comprises a step of testing the location of a probe for intratumoral administration prior to administering the composition. The testing of the location of the probe can comprise intratumorally administering a test injection via the probe and measuring the intratumoral pressure during administration of the test injection. In some instances, the method comprises re-locating the probe when increased or decreased intratumoral pressure is detected during the test injection as compared to pressure of the surrounding tumor tissue. For example, increased pressure can be indicative that the probe is within scar tissue and decreased pressure can be indicative that the probe is within a vessel.

During treatment, a skilled practitioner can use a system, e.g., a computer system, computational unit, software and/or algorithm; to plan, target, position, deliver, monitor, adjust, image, and/or test a treatment protocol. A skilled practitioner would understand that each ablation method involves a number of parameters and variables that can be adjusted and could use an algorithm to control and design the ablation. Any algorithm known in the art can be used in the methods described herein. Examples of computer systems, computational units, software and/or algorithms for use in ablation techniques are known in the art.

Depending on the ablation methods used, the ablation step can be carried out by the ablation techniques and systems known in the art. The discussions below provide non-limiting examples of various ablation methods and devices.

For instance, cryoablation can be carried out by methods and devices described in PCT Application Publication Nos. WO 2004/086936 and WO 2008/142686; U.S. Pat. Nos. 6,074,412; 6,579,287; 6,648,880; 6,875,209; 7,220,257; and 7,001,378; all of which are incorporated herein by reference in their entirety. Exemplary devices include the Endocare™ CryoCare® series, for instance, CryoCare™ and CryoCare CN2 (HealthTronics, Inc., Austin, Tex.); CryoCor™ Cardiac Cryoablation System (CryoCor Inc., Natick, Mass.); Arctic Front® Cardiac CryoAblation Catheter System (Medtronic, Minneapolis, Minn.).

Radio frequency (RF) ablation can be carried out by methods and devices described in U.S. Pat. Nos. 5,246,438; 5,540,681; 5,573,533; 5,693,078; 6,932,814; and 8,152,801; all of which are incorporated herein by reference in their entirety.

Microwave ablation can be carried out by methods and devices described in U.S. Pat. Nos. 6,325,796; 6,471,696; 7,160,292; 7,226,446; and 7,301,131; and U.S. Application Publication No. US 2003/0065317; all of which are incorporated herein by reference in their entirety.

Laser, photo, or plasma ablation can be carried out by methods and devices described in U.S. Pat. Nos. 4,785,806; 5,231,047; 5,487,740; 6,132,424; 8,088,126; 9,204,918; and 10,023,858; and U.S. Application Publication No. US 2007/0129712; all of which are incorporated herein by reference in their entirety.

Ultrasound ablation can be carried out by methods and devices described in U.S. Pat. Nos. 5,342,292; 6,821,274; 7,670,335; and 8,974,446; and U.S. Application Publication Nos. US 2006/0052706 and US 2009/00184; all of which are incorporated herein by reference in their entirety.

High-intensity focused ultrasound (HIFU) ablation can be carried out by methods and devices described in U.S. Pat. Nos. 6,488,639; 6,936,046; 7,311,701; and 7,706,882; and U.S. Application Publication No. US 2008/0039746; all of which are incorporated herein by reference in their entirety.

Steam ablation can be carried out by methods and devices described in U.S. Pat. Nos. 6,813,520 and 9,345,532; and U.S. Application Publication No. US 2013/0178910; all of which are incorporated herein by reference in their entirety.

Reversible electroporation (RE) ablation can be carried out by methods and devices described in U.S. Application Publication Nos. US 2010/0023004 and US 2012/0109122; which are incorporated herein by reference in their entirety.

Irreversible electroporation (IRE) ablation can be carried out by methods and devices described in U.S. Pat. Nos. 7,655,004 and 8,048,067; PCT Application Publication No. WO2012071526; and U.S. Application Publication Nos. US 2012/0109122 and US 2013/0253415; all of which are incorporated herein by reference in their entirety.

Radiofrequency electrical membrane breakdown ablation can be carried out by methods and devices described in U.S. Patent Application US 2015/0150618, PCT Application Publication Nos. WO 2015/085162, WO 2016/123608, WO 2016/127162, WO 2016/126905, WO 2016/126778, and WO 2016/126811; which are incorporated herein by reference in their entirety.

Ablation methods with ultra-short electrical pulse can be carried out by methods and devices described in U.S. Pat. No. 8,926,606; and U.S. Application Publication Nos. US 2006/0056480, US 2010/0261994, and US 2018/015414; all of which are incorporated herein by reference in their entirety. Exemplary devices include the Nano-Pulse Stimulation™ device (Pulse Biosciences, Inc., Hayward, Calif.).

Ablation methods using photodynamic therapy can be carried out by methods and devices described in U.S. Pat. Nos. 6,811,562; 7,996,078; and 8,057,418; all of which are incorporated herein by reference in their entirety.

Ablation methods using non-thermal shock waves can be carried out by methods and devices described in U.S. Pat. Nos. 5,524,620 and 8,556,813; U.S. Application Publication Nos. US 2016/0008016; and Japanese Application No. JP2009061083; all of which are incorporated herein by reference in their entirety.

Ablation with chemical and/or biologics can be carried out by methods and devices described in U.S. Pat. No. 6,428,968; PCT Application Publication Nos. WO 2004/035110; WO 2006/095330, WO 2007/093036, and WO 2014/070820; and U.S. Application Publication Nos. US 2004/0002647, US 2005/0255039, US 2009/0192505, US 2010/0178684, US 2010/0145304, US 2012/0253192; US 2012/0046656, US 2016/0310200, and US 2016/0074626; all of which are incorporated herein by reference in their entirety.

Any of the above ablation techniques and devices can be combined to achieve the desired ablation. For instance, when it is desirable to combine cryoablation with RF-EMB ablation, the methods and device can be modified or combined.

Additionally, the administration of the pharmaceutical composition can also be achieved by the ablation device. For instance, when the pharmaceutical composition is injected to the subject, the injection device can be a cryoprobe. In some instances, the cryoprobe can emit electric pulses and can also deliver plasmids.

Additional descriptions relating to various devices that can combine cryoablation, electroporation, and/or RF-EMB are described in detail in PCT Application Publication No. WO 2017/123981, which is incorporated herein by reference in its entirety. More detailed description regarding using a multi-purpose probe as cryoprobes and/or electrodes are also described in WO 2017/123981.

As used herein, the term “nucleic acid drug” or “therapeutic nucleic acid” refers to a nucleotide, nucleoside, oligonucleotide or polynucleotide that is used to achieve a desired therapeutic effect. Exemplary nucleic acid drugs include, e.g., DNA, nDNA, mtDNA, gDNA, RNA, siRNA, miRNA, mRNA, piRNA, antisense RNA, snRNA, snoRNA, vRNA, etc. For example, the nucleic acid drug can be a DNA plasmid.

The term “subject” is used throughout the specification to describe an animal, human or non-human, to whom treatment according to the methods of the present invention is provided. Veterinary applications are clearly anticipated by the present invention. The term includes but is not limited to birds, reptiles, amphibians, and mammals, e.g., humans, other primates, pigs, rodents such as mice and rats, rabbits, guinea pigs, hamsters, cows, horses, cats, dogs, sheep and goats. Preferred subjects are humans, farm animals, and domestic pets such as cats and dogs. The term “treat(ment),” is used herein to denote delaying the onset of, inhibiting, alleviating the effects of, or prolonging the life of a patient suffering from, a condition, e.g., cancer.

An “effective amount” is an amount sufficient to effect beneficial or desired results. For example, a therapeutically effective amount is one that achieves the desired therapeutic effect or to promote the desired physiological response. Effective amounts of compositions described herein for use in the present invention include, for example, amounts that enhance the immune response against tumors and/or tumor cells, improve the outcome for a patient suffering from or at risk for cancer, and improve the outcome of other cancer treatments. An effective amount can be administered in one or more administrations, applications or dosages. A therapeutically effective amount of a pharmaceutical composition (i.e., an effective dosage) depends on the pharmaceutical composition selected. A therapeutically effective amount of a pharmaceutical composition depends on the method of administration selected. In some cases, intra-tumoral administration of a composition reduces the therapeutically effective amount of a composition, when compared to intraveneous administration (e.g., conventional IV administration). The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the pharmaceutical compositions described herein can include a single treatment or a series of treatments.

The ablation methods can be used alone or in combination with other methods for treating cancer in patients. Accordingly, in some instances, the methods described herein can further include treating the patient using surgery (e.g., to remove a portion of the tumor), chemotherapy, immunotherapy, gene therapy, and/or radiation therapy. Compositions and methods described herein can be administered to a patient at any point, e.g., before, during, and/or after the surgery, chemotherapy, immunotherapy, gene therapy, and/or radiation therapy.

The pharmaceutical compositions and treatment methods described herein are particularly useful for treating cancer in subjects. The term “cancer” refers to cells having the capacity for autonomous growth. Examples of such cells include cells having an abnormal state or condition characterized by rapidly proliferating cell growth. The term is meant to include cancerous growths, e.g., tumors; metastatic tissues, and malignantly transformed cells, tissues, or organs, irrespective of histopathologic type or stage of invasiveness. Also included are malignancies of the various organ systems, such as respiratory, cardiovascular, renal, reproductive, hematological, neurological, hepatic, gastrointestinal, and endocrine systems; as well as adenocarcinomas which include malignancies such as most colon cancers, renal-cell carcinoma, prostate cancer and/or testicular tumors, non-small cell carcinoma of the lung, cancer of the small intestine, and cancer of the esophagus.

The pharmaceutical compositions and treatment methods described herein can be used to treat naturally arising cancer in a subject. Cancer that is “naturally arising” includes any cancer that is not experimentally induced by implantation of cancer cells into a subject, and includes, for example, spontaneously arising cancer, cancer caused by exposure of a patient to a carcinogen(s), cancer resulting from insertion of a transgenic oncogene or knockout of a tumor suppressor gene, and cancer caused by infections, e.g., viral infections.

Cancers to be treated with the pharmaceutical compositions and treatment methods described herein also include carcinomas, adenocarcinomas, and sarcomas. The term “carcinoma” is art recognized and refers to malignancies of epithelial or endocrine tissues. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

Cancers or tumors that may be treated using the treatment methods and pharmaceutical compositions described herein include, for example, cancers or tumors of the stomach, colon, rectum, mouth/pharynx, esophagus, larynx, liver, pancreas, lung, breast, cervix uteri, corpus uteri, ovary, prostate, testis, bladder, skin, bone, kidney, brain/central nervous system, head, neck, thyroid, and throat; sarcomas, choriocarcinomas, and lymphomas, among others. Exemplary tumors or cancers to be treated are cancers or tumors of prostate, pancreas, colon, lung, and bladder.

Metastatic tumors or cancers (Stage IV) can be treated using the treatment methods and pharmaceutical compositions described herein. For example, performing a treatment method described herein on a tumor or cancer located at one site in the subject's body (e.g., a primary tumor), can stimulate the subject's immune defenses against the tumor or cancer and cause an immune attack on tumors or cancers of the same or even different type of at another site(s) in the subject's body (e.g., a metastatic tumor). A metastatic tumor or cancer can arise from a multitude of primary tumor or cancer types, including but not limited to, those of brain, prostate, colon, lung, breast, bone, peritoneum, adrenal gland, muscle, and liver origin. Metastases develop, e.g., when tumor cells shed from a primary tumor adhere to vascular endothelium, penetrate into surrounding tissues, and grow to form independent tumors at sites separate from a primary tumor.

Skilled practitioner will appreciate that the treatment methods and pharmaceutical compositions described herein can also be used to treat other stages of cancers or tumors, such as carcinoma in situ (stage 0), localized early stage cancer (stage I), and larger tumors or cancers (stage II and stage III).

Skilled practitioners will appreciate that the pharmaceutical compositions and treatment methods described herein can also be used to treat non-cancerous growths, e.g., noncancerous tumors. Exemplary non-cancerous growths include, e.g., benign tumors, adenomas, adenomyoeptheliomas, ductal or lobular hyperplasia, fibroadenomas, fibromas, fibrosis and simple cysts, adenosis tumor, hematomas, hamartomas, intraductal papillomas, papillomas, granular cell tumors, hemangiomas, lipomas, meningiomas, myomas, nevi, osteochondromas, phyllodes tumors, neuromas (e.g., acoustic neuromas, neurofibromas, and pyogenic granulomas), or warts (e.g., plantar warts, genital warts, flat warts, periungual warts, and filiform warts).

Skilled practitioners will appreciate that a subject can be diagnosed by a physician (or veterinarian, as appropriate for the subject being diagnosed) as suffering from or at risk for a condition described herein, e.g., cancer, by any method known in the art, e.g., by assessing a patient's medical history, performing diagnostic tests, and/or by employing imaging techniques.

As described herein, one exemplary method of treating a tumor in a patient comprises the steps of: (i) optionally, prior to performance of the method, identifying the location of the tumor or cancer within the patient; (ii) intratumorally administering a pharmaceutical composition described herein to the tumor or cancer (e.g., a pharmaceutical composition comprising at least two immune checkpoint inhibitors and at least one cytotoxic or cytostatic chemotherapeutic drug); (iii) optionally ablating at least a portion of the tumor; and (iv) optionally administering a therapeutically effective amount of a nucleic acid drug to the tumor.

Identifying a location of the tumor can be performed by techniques known in the art (e.g., X-ray radiography, magnetic resonance imaging, medical ultrasonography or ultrasound, endoscopy, elastography, tactile imaging, thermography, medical photograph, nuclear medicine imaging techniques including positron emission tomography and single-photon emission computed tomography, photoacoustic imaging, thermography, tomography including computer-assisted tomography, echocardiography and functional near-infrared spectroscopy, etc.). The optional step of ablating the tumor (iii) can occur before, concurrently, or after administering a pharmaceutical composition (ii), and the ablation can create an ablation site exposing intracellular components and membrane antigens of the tumor. Ablation can be performed using a technique described herein on a portion or all of the tumor. Optionally administering a therapeutically effective amount of a nucleic acid drug to the tumor (iv) can occur before, concurrently or after the of steps (ii) and (iii).

Also provided are kits that include one or more of the pharmaceutical compositions described herein. Kits generally include the following major elements: packaging, reagents comprising binding compositions as described above, optionally a control, and instructions. Packaging can be a box-like structure for holding a vial (or number of vials) containing said binding compositions, a vial (or number of vials) containing a control, and/or instructions for use in a method described herein. In some cases the packaging contains a cartridge that can be controlled by a digital device following systematic instructions. Individuals skilled in the art can readily modify the packaging to suit individual needs.

In some embodiments, a kit provided herein can include at least one (e.g., one, two, three, four, five, or more) composition containing at least one (e.g., one, two, three, four, five, or more) of the compositions described herein, and at least one (e.g., one, two, three, four, five, or more) other composition in a separate vial containing a therapeutic or biologic agent known in the art to be effective in treating cancer.

Compositions and kits as provided herein can be used in accordance with any of the methods (e.g., treatment methods) described above. For example, compositions and kits can be used to treat cancer or tumor. Those skilled in the art will be aware of other suitable uses for compositions and kits provided herein, and will be able to employ the compositions and kits for such uses.

### EXAMPLES

The following examples are given as particular embodiments of the invention and to demonstrate the practice and advantages thereof. It is to be understood that the examples are given by way of illustration and are not intended to limit the specification or the claims that follow in any manner.

Preliminary results of selected patients in a Phase II clinical trial, consistent with the protocol listed under “A Phase 2 Trial for Men With Metastatic Prostatic Adenocarcinoma,” NCT04090775, in the database of ClinicalTrials.gov (the details of the protocol listed in NCT04090775 are incorporated herein by reference in its entirety) are shown in the following examples. Patients have been treated with a combination of intra-prostatic (intra-tumoral), temperature-limited cryosurgery and immunologic and chemotherapeutic agents with at least 2 months of follow-up treatment, as described in the examples below.

### Example 1: Treatment of Prostate Cancer in Patient a Using a Combination of Cryoablation and Immunologic and Chemotherapeutic Medications

A 60 year-old man presented with severe bone pain which was diagnosed as widespread skeletal and spine metastases from high-grade prostate cancer. He was not considered a surgical candidate, so he received radiation therapy as well as hormonal therapy using the standard cancer medicines. Despite this treatment, he reported that the pain persisted. Over the course of a year, the metastases enlarged and progressed to involve additional sites.

The patient underwent two rounds of treatments, at an eight-week interval, to his prostate. For each treatment the patient received, a temperature-limited cryoablation (a cryosurgical freezing) to his prostate (intra-prostatic, intra-tumoral) was carried out at a temperature about −40° C. with a duration of about 4 minutes. This was immediately followed, at the same site, by intra-tumoral injection of a composition comprising a CTLA-4 inhibitor (ipilimumab, 5 mg/ml, 1.0 ml), a PD-1 inhibitor (nivolumab, 10 mg/ml, 1.0 ml), and a low-dose chemotherapeutic agent (cyclophosphamide, 50 mg/ml, 1.0 ml).

The patient reported that his bone pain was “ . . . much improved” following treatment, and that this improvement has persisted for more than 6 months. Also, his radiologist found that the patient's ¹⁸F PET-CT whole-body bone scan showed “ . . . considerable improvement in lumbar spinal, pelvic, and left femoral metastases . . . [and] slight improvement in the remaining metastases involving the skull base, cervical and thoracic spine, bilateral ribs, clavicles, and bilateral humoral bones.”

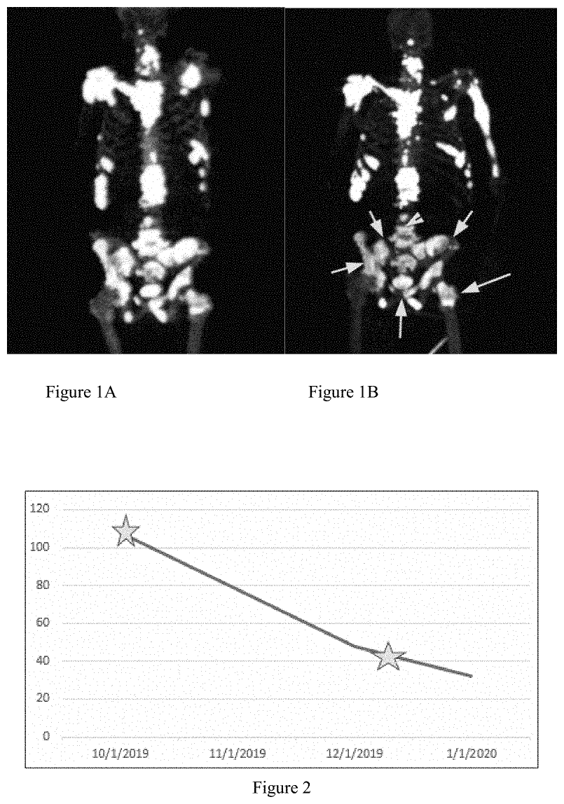

FIGS. 1A-1B are images of FDG-PET (¹⁸F-fluorodeoxyglucose positron emission tomography) scans of Patent A's whole body bone before (FIG. 1A) and about 3 months after (FIG. 1B) treatment discussed in this example. Comparison of the scans before FIG. 1A) and about 3 months after (FIG. 1B) treatment reveals considerable improvement in bone metastases; the arrows point to the region (the pelvis region) that the improvements are most prominent. Analyzing the results of FDG-PET show that activity in the right shoulder after the treatment had maximal SUV (standardized uptake value) of 34.6, as compared to previous maximal SUV of 55.6 prior to the treatment, a decrease of about 38%.

This data indicates a “good outcome,” and that the patient had a “response” to the treatment, according to the PET Response Criteria in Solid Tumors (PERCIST) 1.0 criteria describing that, in clinical trials, medically-relevant beneficial changes indicating “response” are characterized by a decline of 30% or more in tumor standardized uptake value (SUV), and larger drops in tumor SUV of more than 30-35% are associated with a good outcome. See Wahl et al., “From RECIST to PERCIST: Evolving Considerations for PET Response Criteria in Solid Tumors,” J. Nucl. Med. 50 (suppl. 1): 122S-150S (2009), which is incorporated herein by reference in its entirety. More descriptions relating to FDG-PET, SUV and its determination, and PET criteria relating to cancer treatment response can be found in Wahl et al.

There were no significant adverse events.

### Example 2: Treatment of Prostate Cancer in Patient B Using a Combination of Cryoablation and Immunologic and Chemotherapeutic Medications

A 62 year-old man was diagnosed with elevated serum prostate-specific antigen (PSA) concentration was found to have high-grade prostate cancer with metastases to retroperitoneal lymph nodes and pelvic bones. He was treated with hormonal therapy using the basic and advanced 2^{nd} line cancer medicines. The treatment was unsuccessful, and, after five years, the cancer was categorized as castrate-resistant.

The patient underwent two rounds of treatments, at an eight-week interval, to his retroperitoneal lymph nodes. For each treatment the patient received, a temperature-limited cryoablation (a cryosurgical freezing) to his retroperitoneal lymph nodes was carried out at a temperature about −40° C. with a duration of about 4 minutes. This was immediately followed, at the same site, by intra-tumoral injection of a composition comprising a CTLA-4 inhibitor (ipilimumab, 5 mg/ml, 1.0 ml), a PD-1 inhibitor (nivolumab, 10 mg/ml, 1.0 ml), and a low-dose chemotherapeutic agent (cyclophosphamide, 50 mg/ml, 1.0 ml).

Within 3 months, the retroperitoneal cancer had shrunken in volume by 57%, which indicates a rapid and significant “partial response,” according to the World Health Organization criteria and the Response Evaluation Criteria in Solid Tumors (RECIST) 1.1 criteria describing that, in clinical trials, a decline of at least 30% in tumor diameters for at least 4 weeks would be considered as a “partial response.” See Wahl et al., “From RECIST to PERCIST: Evolving Considerations for PET Response Criteria in Solid Tumors,” J. Nucl. Med. 50 (suppl. 1): 122S-150S (2009). The next category is a “complete response” which requires the disappearance of all tumor foci for at least 4 weeks.

FIG. 2 is a graph showing the results of Patient B's serum prostate-specific antigen (PSA) concentrations following two rounds of treatments discussed in this example. PSA, is a protein produced by cells of the prostate gland. The blood level of PSA is often elevated in men with prostate cancer, with a level of 10 ng/ml higher indicative of the patient having at least 50% chance of having prostate cancer. As illustrated in the figure, the serum PSA has shown a significant decline from 107.6 ng/mL to 31.9 ng/mL (about 70% decline) following two treatments.

There were no significant adverse events.

What is claimed is:

1. A pharmaceutical composition, comprising at least two immune checkpoint inhibitors, at least one cytotoxic or cytostatic chemotherapeutic drug, and optionally a pharmaceutically acceptable carrier.

2. The pharmaceutical composition of claim 1, wherein the immune checkpoint inhibitors are different and are each inhibitors of an immune checkpoint molecule selected from the group consisting of CD137, CD134, PD-1, KIR, LAG-3, PD-L1, PDL2, CTLA-4, B7.1, B7.2, B7-DC, B7-H1, B7-H2, B7-H3, B7-H4, B7-H5, B7-H6, B7-H7, BTLA, LIGHT, HVEM, GAL9, TIM-3, TIGHT, VISTA, 2B4, CGEN-15049, CHK 1, CHK2, A2aR, TGF-β, PI3Kγ, GITR, ICOS, IDO, TLR, IL-2R, IL-10, PVRIG, CCRY, OX-40, CD160, CD20, CD52, CD47, CD73, CD27-CD70, and/or CD40.

3. The pharmaceutical composition of claim 2, wherein the at least two immune checkpoint inhibitors comprise i) a CTLA-4 inhibitor and ii) a PD-1 inhibitor or PD-L1 inhibitor.

4. The pharmaceutical composition of claim 3, wherein the CTLA-4 inhibitor is ipilimumab, tremelimumab, or a combination thereof.

5. The pharmaceutical composition of claim 3, wherein the PD-1 inhibitor is selected from the group consisting of pembrolizumab, nivolumab, pidilizumab, MK-3475, MED 14736, CT-011, spartalizumab, and combinations thereof.

6. The pharmaceutical composition of claim 3, wherein the PD-L1 inhibitor is selected from the group consisting of durvalumab, atezolizumab, avelumab, AMP224, BMS-936559, MPLDL3280A, MSB0010718C, and combinations thereof.

7. The pharmaceutical composition of claim 3, wherein the at least two immune checkpoint inhibitors comprise a CTLA-4 inhibitor and a PD-1 inhibitor; and wherein the CTLA-4 inhibitor is ipilimumab and the PD-1 inhibitor is pembrolizumab or nivolumab.

8. The pharmaceutical composition of claim 1, wherein the cytotoxic or cytostatic chemotherapeutic drug is selected from the group consisting of asparaginase, bleomycin, busulphan, carboplatin, cetuximab, cisplatin, cyclophosphamide, BCG, chloramphenicol, colchicine, cyclosporin, dacarbazine, doxorubicin, etoposide, fludarabine, gemcitabine, ifosfamide, irinotecan, lomustin, melphalan, methotrexate, mitomycin, mitoxantrone, paclitaxel, procarbazine, rituximab, temozolomide, thitepa, vinblastine, vincristine, zidovudine, and combinations thereof.

9. The pharmaceutical composition of claim 1, further comprising a second cytotoxic or cytostatic chemotherapeutic drug.

10. The pharmaceutical composition of claim 3, wherein the concentration of the CTLA-4 inhibitor ranges from about 0.5 to about 10 mg/ml, and the concentration of the PD-1 or the PD-L1 inhibitor ranges from about 0.5 to about 20 mg/ml.

11. The pharmaceutical composition of claim 1, wherein the immune checkpoint inhibitors and the cytotoxic or cytostatic chemotherapeutic drug are formulated for intratumoral administration.

12. The pharmaceutical composition of claim 1, further comprising one or more nucleic acid drugs.

13. The pharmaceutical composition of claim 12, wherein the nucleic acid drug is a DNA plasmid.

14. The pharmaceutical composition of claim 12, wherein the DNA plasmid comprises a nucleotide sequence encoding a gene selected from the group consisting of GM-CSF, IL-12, IL-6, IL-4, IL-12, TNF, IFNγ, IFNα, and combinations thereof.

15. A method of treating a tumor or a cancer in a patient comprising:
administering to a patient in need thereof a composition comprising: at least two immune checkpoint inhibitors and at least one cytotoxic or cytostatic chemotherapeutic drug, in an amount effective to treat the tumor or cancer.

16. The method of claim 15, wherein the composition is administered to the patient intratumorally.

17. The method of claim 15, wherein the composition is administered to the patient's tumor or cancer using an injection device.

18. The method of claim 15, wherein the immune checkpoint inhibitors are different and are each inhibitors of an immune checkpoint molecule selected from the group consisting of CD137, CD134, PD-1, KIR, LAG-3, PD-L1, PDL2, CTLA-4, B7.1, B7.2, B7-DC, B7-H1, B7-H2, B7-H3, B7-H4, B7-H5, B7-H6, B7-H7, BTLA, LIGHT, HVEM, GALS, TIM-3, TIGHT, VISTA, 2B4, CGEN-15049, CHK 1, CHK2, A2aR, TGF-β, PI3Kγ, GITR, ICOS, IDO, TLR, IL-2R, IL-10, PVRIG, CCRY, OX-40, CD160, CD20, CD52, CD47, CD73, CD27-CD70, and/or CD40.

19. The method of claim 18, wherein the at least two immune checkpoint inhibitors comprise i) a CTLA-4 inhibitor and ii) a PD-1 inhibitor or PD-L1 inhibitor.

20. The method of claim 15, wherein the cytotoxic or cytostatic chemotherapeutic drug is selected from the group consisting of asparaginase, bleomycin, busulphan, carboplatin, cetuximab, cisplatin, cyclophosphamide, BCG, chloramphenicol, colchicine, cyclosporin, dacarbazine, doxorubicin, etoposide, fludarabine, gemcitabine, ifosfamide, irinotecan, lomustin, melphalan, methotrexate, mitomycin, mitoxantrone, paclitaxel, procarbazine, rituximab, temozolomide, thitepa, vinblastine, vincristine, zidovudine, and combinations thereof.

21. The method of claim 19, wherein the concentration of the CTLA-4 inhibitor ranges from about 0.5 to about 10 mg/ml, and the concentration of the PD-1 or the PD-L1 inhibitor ranges from about 0.5 to about 20 mg/ml.

22. The method of claim 15, further comprising administering one or more therapeutically effective amount of nucleic acid drugs to the tumor or cancer.

23. The method of claim 22, wherein the nucleic acid drug is a DNA plasmid comprising a nucleotide sequence encoding a gene selected from the group consisting of GM-CSF, IL-12, IL-6, IL-4, IL-12, TNF, IFNγ, IFNα, and combinations thereof.

24. The method of claim 15, further comprising a step of ablating at least a portion of the tumor or cancer.

25. The method of claim 24, wherein the ablating step is conducted before or at the same time as the administering step.

26. The method of claim 24, wherein the ablating step is carried out by cryoablation; radio frequency (RF) ablation; microwave ablation; laser, photo, or plasma ablation; ultrasonic ablation; high-intensity focused ultrasound (HIFU) ablation; steam ablation; reversible electroporation (RE); irreversible electroporation (IRE); radiofrequency electrical membrane breakdown (RF-EMB); RF-EMB type ablation; ablation with ultra-short electrical pulse; ablation using photodynamic therapy; ablation using non-thermal shock waves; cavitation; other mechanical physical means to create cell disruption; chemical ablation; ablation with biologics; or combinations thereof.

27. The method of claim 26, wherein the ablating step is carried out by cryoablation, and/or RF-EMB.

28. The method of claim 27, wherein the ablating step is carried out by cryoablation in a minimally invasive manner.

29. The method of claim 28, wherein the ablating step is carried out using a single probe, with total ablating time of no more than 5 minutes.

30. The method of claim 29, wherein the cryoablation is carried out using a single probe with a diameter of no more than 1 mm.

31. The method of claim 28, wherein the cryoablation is carried out at a temperature from about −35 to about −45° C.

32. The method of claim 25, wherein the step of administering the composition and the ablating step are carried out using a same device that comprises an ablation module and an injection module.

33. The method of claim 15, wherein the tumor or cancer type is selected from the group consisting of prostate, pancreatic, colon, lung, and bladder.

34. The method of claim 15, wherein the tumor or cancer is metastatic.

35. The pharmaceutical composition of claim 1, wherein the cytotoxic or cytostatic chemotherapeutic drug is cyclophosphamide.

36. The method of claim 20, wherein the cytotoxic or cytostatic chemotherapeutic drug is cyclophosphamide.
